(19) United States
(12) Patent Application Publication
(10) Pub. No.: US 2019/0216751 A1
(43) Pub. Date: Jul. 18, 2019

(54) Compositions and Methods for the Treatment and Prevention of Cancer

(71) Applicant: California State University Northridge, Northridge, CA (US)

(72) Inventors: Jonathan A Kelber, Northridge, CA (US); Yvess Adamian, Northridge, CA (US); Lindsay Kutscher, Northridge, CA (US); Robert Guth, Northridge, CA (US); Cameron Geller, Northridge, CA (US)

(73) Assignee: California State University Northridge, Northridge, CA (US)

(21) Appl. No.: 16/188,821

(22) Filed: Nov. 13, 2018

Related U.S. Application Data
(60) Provisional application No. 62/586,632, filed on Nov. 15, 2017.
(60) Provisional application No. 62/642,511, filed on Mar. 13, 2018.

(51) Int. Cl. A61K 31/155; A61K 31/422; A61K 31/517; A61K 31/17; A61K 31/437; A61K 31/166; A61K 31/343; A61K 31/5377; A61K 31/506; A61P 35/00

(52) U.S. Cl. CPC A61K 31/155; A61K 31/422; A61K 31/517; A61K 31/17; A61P 35/00; A61K 31/166; A61K 31/343; A61K 31/5377; A61K 31/506; A61K 31/437

(57) ABSTRACT

A method of treating a subject with cancer is provided. The method includes administering to a subject in need thereof an effective amount of a pharmaceutical composition that includes inhibitors of HDAC6 and/or sirtuins and an inhibitor of deoxyhypusine synthase (DHPS). Methods also include treating a subject with cancer by administering to a subject in need thereof an effective amount of a pharmaceutical composition comprising at least one inhibitor of sirtuin and at least one inhibitor of histone deacetylase six (HDAC6); at least one inhibitor of sirtuin or at least one inhibitor of histone deacetylase six (HDAC6), or a combination thereof. Additional methods include treating a subject with cancer by administering to a subject in need thereof an effective amount of a pharmaceutical composition comprising at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof.

This United States Utility Application claims priority to U.S. Provisional Application Ser. No.: 62/586,632 filed on Nov. 15, 2017 and U.S. Provisional Application Ser. No.: 62/642,511 filed on Mar. 13, 2018, both of which are incorporated herein in their entirety by reference.

### STATEMENT REGARDING FEDERALLY SPONSORED RESEARCH OR DEVELOPMENT

This subject matter was made with government support under National Institutes of Health grant 1SC1GM121182. The government has certain rights in this subject matter.

### FIELD OF THE SUBJECT MATTER

The subject matter disclosed herein relates to compositions and methods for the treatment and prevention of cancer.

### BACKGROUND

Breast cancer is the most commonly diagnosed cancer among women in the United States, but it remains the second leading cause of cancer related deaths¹. Percent survival is nearly 100% if the cancer is diagnosed at stage 1 (the localized stage). If the tumor is diagnosed at stage 2 and 3, the patient's tumor has disseminated to local lymph nodes and adjacent tissue. Still, the patient has approximately an 80% survival rate. At stage 4; however, the percent survival plummets to about 20% because the cancer has invaded into the vasculature and metastasized to distant organs such as bones, liver, lung and brain^{2,3}. Although the overall weighted average is about 85%, the majority of these deaths are due to the metastatic form of this disease. Thus, there is a need to understand the molecular mechanisms regulating metastasis to provide therapeutic targets and increase patient survival.

The pseudopodium is an actin-rich structure that protrudes from the cell surface and drives cancer cell migration/invasion away from the primary tumor⁴. During cancer progression, cells use specialized pseudopodia, known as invadopodia, to invade into and out of the bloodstream during metastasis. To learn more about the protein profile of this subcellular structure, the inventors plated mammalian cells onto a porous membrane and exposed them to a chemoattractant. This promoted pseudopodia migration through the pores, enabling their easy mechanical separation from the cell body. Using mass spectrometry, 819 pseudopodium-enriched proteins were identified⁵. Since tyrosine phosphorylation of cytoskeleton-associated proteins plays a central role in cell signaling, pseudopodium formation and cancer metastasis, phosphotyrosine immunoaffinity purification followed by Multidimensional Protein Identification Technology (MudPIT) was used to identify kinase targets and potentially novel anti-cancer targets. One hundred thirty of the 819 proteins were determined to be phospho-tyrosine (pY) proteins. A few of these pY proteins enriched in the pseudopodium had not been previously cloned or studied. One of those proteins, later named PEAK1 (or pseudopodium-enriched atypical kinase 1), was determined to be enriched by 2.6 fold in the pseudopodium. Notably, PEAK1 has many predicted phosphorylation sites and interactions with well-established tumor promoting signaling pathways^{4,6}. The molecular weight of PEAK1 is approximately 190 kD and it is a non-receptor tyrosine kinase. It is also one of the two members in the New Kinase Family Three (NKF3)⁷. PEAK1 promotes tumor growth, metastasis and therapy resistance in human cancers via its regulation of the actin cytoskeleton and Src, KRas and ErbB2 signaling^{4,6,8}. PEAK1 overexpression in non-malignant and malignant human mammary epithelial cells induces epithelial to mesenchymal transition (EMT), a prerequisite for solid tumor metastasis, via its regulation of fibronectin/TGFβ signaling^{9,10,11}. PEAK1 regulates Shc1 signaling by interacting with Grb2 and MAPK controlling cell morphology, movement and proliferation downstream of EGF signaling¹². Finally, it was demonstrated that hypusination/activation of the eIF5A translation factor can promote PEAK1 protein production and pancreatic cancer progression¹³.

It was previously demonstrated that TGFβ-induced EMT upregulates PEAK1 expression¹⁰. EMT is the gradual loss of epithelial characteristics and the acquisition of mesenchymal or spindle-like characteristics. TGFβ is a well characterized inducer of EMT during normal development and disease progression, and these cells acquire more invasive and migratory behavior.

A recent review from the Massagué lab on the pleiotropic and often opposing roles of TGFβ suggests that when TGFβ binds to its type II receptor (TβRII), recruiting its type I (ALK 5) receptor, phosphorylation of Mother Against Decapentaplegic Homolog 2/3 (SMAD2/3) results in gene transcription of tumor suppressor genes¹⁵. The Schiemann group has also demonstrated that this pathway is induced in the presence of fibronectin/ITGβ3 by activating Src which subsequently phosphorylates TβRII. Grb2 is then recruited and binds to a phospho-tyrosine site on TβRII to then stimulate MAPK signaling, migration, proliferation and therapy resistance. Importantly, this non-canonical TGFβ signaling leads to EMT and metastasis^{15,16,17}. Notably, it was recently reported that in the presence of fibronectin and with increasing expression of PEAK1, TGFβ can switch to a pro-tumorigenic factor. When PEAK1 is upregulated in the presence of fibronectin, ZEB1 expression is induced which causes EMT and metastasis to occur^{10,11,14}.

In relation to this, high PEAK1 levels may indicate cases/conditions where TGFβ will promote cancer progression. Patients with upregulated PEAK1 expression may benefit from anti-TGFβ therapy; however, targeting TGFβ altogether could be detrimental to the patient since TGFβ could also act as a tumor-suppressive cytokine in other parts of the body. Eukaryotic Initiation Factor 5A (eIF5A) is involved in translation of proteins carrying a unique post-translational modification termed hypusine at lysine residue 50. Its role in the process of hypusination is spermidine dependent and is carried out in two subsequent steps involving the activity of two enzymes: deoxyhypusine synthase (DHPS) and deoxyhypusine hydroxylase (DOHH). Once eIF5A is in its active form, translation of PEAK1 mRNA into protein can occur, as well as tumor progression. Currently, there are two drugs available to target the pathway in which eIF5A becomes hypusinated/activated. N1-Guanyl-1,7-diaminoheptane (GC7)—a selective inhibitor of DHPS and Ciclopirox olomine (CPX)—an iron chelator that reduces the activity of DOHH¹⁴. To this end, eIF5A hypusination/activation may be targeted in breast cancer patients that exhibit elevated levels of PEAK1.

Using an online interactive database called The Human Protein Atlas, various tissue-specific proteomes can be explored using real human patient tissue samples. The expression pattern of a mesenchymal cancer patient tissue exhibiting an undifferentiated tumor type shows evidence of this pathway directed towards hypusinated eIF5A such as spermidine synthase, DHPS and DOHH as well as eIF5A1 and eIF5A2¹⁸.

There are contrasting views describing breast cancer response to eIF5A. A recent publication described that inhibiting the activity of a histone deacetylase enzyme 6, HDAC6, results in an increase in eIF5A acetylation (Ishfaq et al., 2012 FEBS Letters). Another publication concluded that TGFβ increases the activity of HDAC6 to promote EMT regulation²⁰. Ishfaq's group reported that eIF5A2 is acetylated at lysine residue at site 47. HDAC6 and SIRTUIN2 are the histone deacetylases responsible for deacetylating eIF5A in order for it to be exported out into the cytoplasm. Ishfaq's group also identified a short crosstalk between acetylation and hypusination that when DHPS and DOHH are prevented from hypusinating eIF5A, a dramatic increase in acetylation level is seen; however, upon deacetylation, eIF5A is hypusinated suggesting a direct link between the lysine residues²¹.

Exportin 4 (XPO4) is a bidirectional nuclear transport receptor that mediates nuclear export of eIF5A and other proteins such as Smad3^{22,23,24}. The hypusine modification found on eIF5A when localized in the nucleus is recognized by XPO4. eIF5A is then allowed access to enter the XPO4 pathway which then can be shuttled out to the cytoplasm. The hypusine modified eIF5A protein residue binds to XPO4 35-times more than eIF5A protein that lacks this modification²⁴.

Furthermore, when eIF5A is transported out into the cytoplasm, its job is to promote translation elongation and termination of proteins not limited to proline stretches²⁵. In a study done by Schuller's group, as previously thought, the hypusine residue is a necessity for polyproline (PPP) rich protein translation; however, during ribosomal pausing, eIF5A alleviates translation not limited to PPP motifs. During a depletion of eIF5A, a study revealed that 188 out of the 972 proteins altered contained at least one PPP motif²⁶. Interestingly, during TβRII inhibition, both eukaryotic initiation and elongation processes significantly decreased.

### SUMMARY OF THE SUBJECT MATTER

One aspect of the present contemplated subject matter is directed to a method of treating a subject with cancer. The method includes administering to a subject in need thereof an effective amount of a pharmaceutical composition that includes at least one inhibitor of histone deacetylase six (HDAC6) and/or an inhibitor of SIRTUIN2 and/or an inhibitor of deoxyhypusine synthase (DHPS).

In one embodiment, the cancer is breast cancer.

In another embodiment, the inhibitor of HDAC6 includes one of the following:

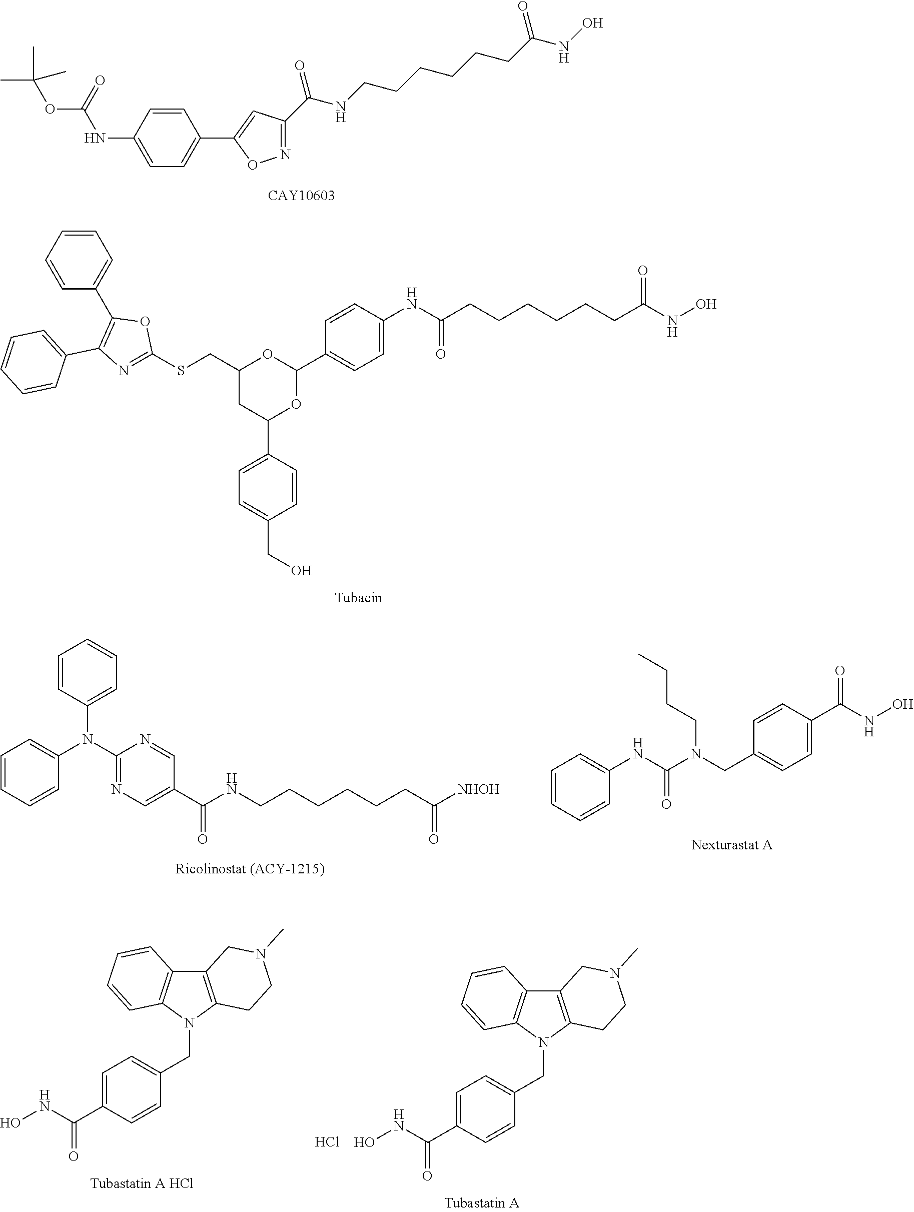

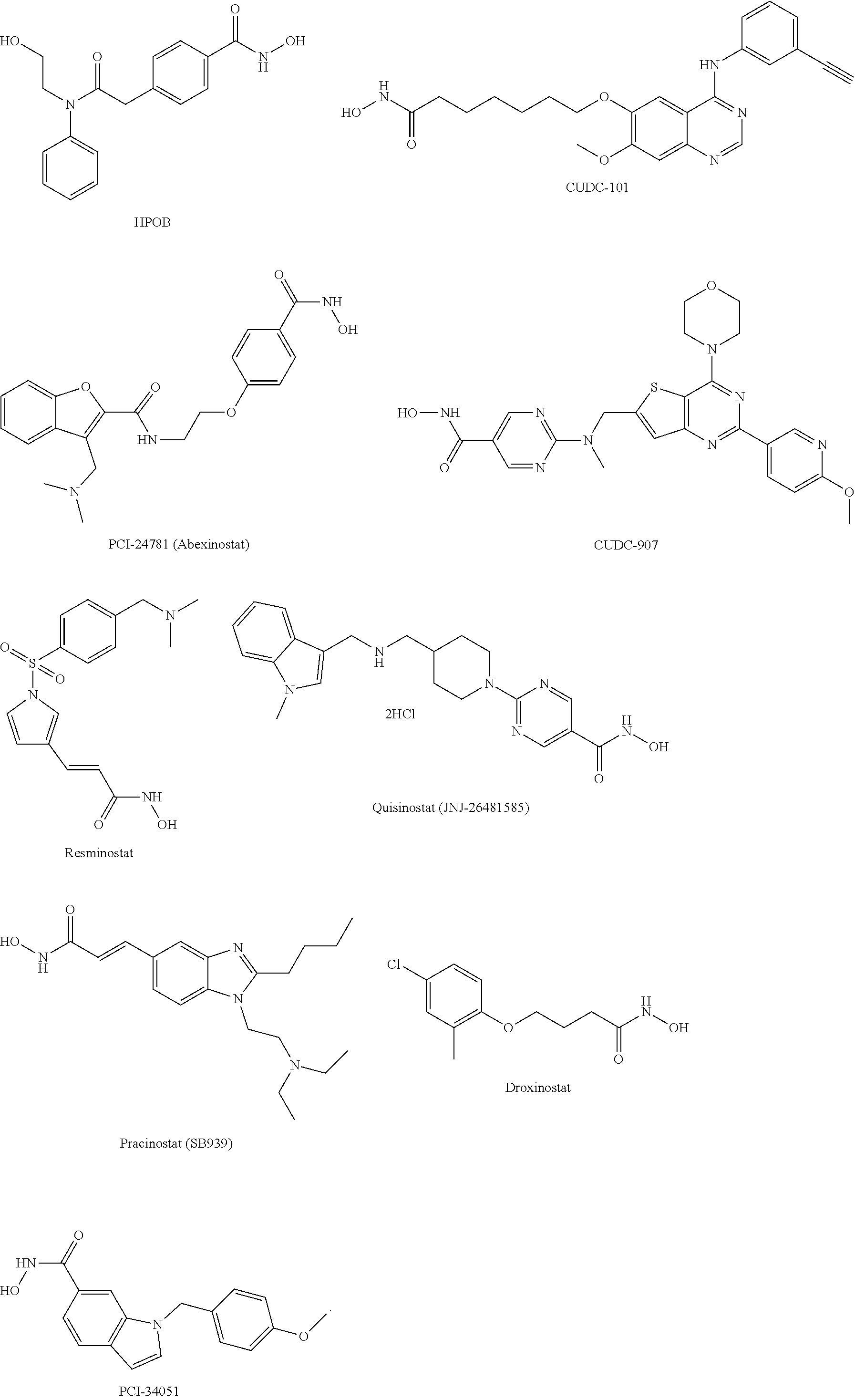

In another embodiment, the inhibitor of DHPS is

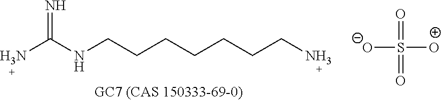 [SMILES: N=C([NH3+])NCCCCCCC[NH3+] ; O=S(=O)([O-])[OH2+]]

In another embodiment, the inhibitor of HDAC6 is Tubastatin A, and the inhibitor of DHPS is GC7 (CAS 150333-69-0).

Another aspect of the present contemplated subject matter is directed to a pharmaceutical composition for the treatment of cancer. The pharmaceutical composition includes an inhibitor of histone deacetylase six (HDAC6) and an inhibitor of deoxyhypusine synthase (DHPS).

In one embodiment, the cancer is breast cancer or pancreatic cancer. Other types of cancer are contemplated herein as well.

In another embodiment, the pharmaceutical composition further includes at least one excipient.

Other aspects and advantages of the contemplated subject matter will be apparent from the following description and the appended claims.

Contemplated herein are methods of treating a subject with cancer comprising administering to a subject in need thereof an effective amount of a pharmaceutical composition comprising at least one inhibitor of sirtuin and at least one inhibitor of histone deacetylase six (HDAC6); at least one inhibitor of sirtuin or at least one inhibitor of histone deacetylase six (HDAC6), or a combination thereof. Pharmaceutical compositions are contemplated herein as well.

Additional contemplated methods include treating a subject with cancer comprising administering to a subject in need thereof an effective amount of a pharmaceutical composition comprising at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof. Pharmaceutical compositions are contemplated herein as well.

A pharmaceutical composition for the treatment of cancer comprises at least one inhibitor of histone deacetylase six (HDAC6) and an inhibitor of deoxyhypusine synthase (DHPS); at least one inhibitor of histone deacetylase six (HDAC6) or an inhibitor of deoxyhypusine synthase (DHPS), or a combination thereof.

A pharmaceutical treatment for treating a subject with cancer comprises at least one inhibitor of sirtuin and at least one inhibitor of histone deacetylase six (HDAC6); at least one inhibitor of sirtuin or at least one inhibitor of histone deacetylase six (HDAC6), or a combination thereof.

A pharmaceutical treatment for treating a subject with cancer comprises at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof.

A composition is contemplated that comprises at least one inhibitor of sirtuin and at least one inhibitor of histone deacetylase six (HDAC6); at least one inhibitor of sirtuin or at least one inhibitor of histone deacetylase six (HDAC6), or a combination thereof.

A composition is contemplated that comprises at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof.

### BRIEF DESCRIPTION OF THE DRAWINGS

FIGS. 1A-1E. eIF5A Isoform Expression Levels Predict Hypusination Profile in Breast Cancer Lines (A) Western blot illustrates protein lysates collected from non-malignant human mammary cells (MCF10A), malignant H-Ras Transformed breast cancer cells (MCF10AT1K, MCF10CA1h and MCF10CA1a), triple negative breast cancer cells (MDA-MB-231 and MDA-MB-468) and mouse malignant breast cancer cells (4T1 and 67NR). Immunoblotting was stained for hypusine, total eIF5A and β-actin as a control. (B) Relative band intensity is graphed using Prism software for Western blot from panel A. Ratios are set relative to MCF10A comparing hypusine to total eIF5A. (C) qPCR for all cell lines showing relative mRNA expression for both isoforms of eIF5A relative to isoform 1. Time-course of experiment is illustrated to the right of panel A. (D and E) IHC patient data from The Human Protein Atlas show proteins from a breast cancer patient exhibiting a mesenchymal phenotype (Scale bar: 100 μm and 50 μm for full image and inlay, respectively).

FIG. 2A-2B. GC7 Decreases Cell Proliferation/Number Across Breast Cancer Cell Lines (A) Cytotoxicity graphs show cell viability graphed relative to control (water). Aqueous One reagent was used 72 hours after CPX and GC7 drug treatments (0.1 nM→1 mM). Time-course of experiment is illustrated to the right of panel A. (B) Phase-contrast micrographs were taken after 48 hours of water or 100 μM GC7 treatment for all cell lines. Time-course of experiment is illustrated to the right of panel B (Scale bar: 100 μm).

FIGS. 3A-3C. GC7 Inhibits eIF5A Hypusination and Cell Number in a Dose-Dependent Manner (A) Western blot illustrates protein lysates from eight all cell lines that were treated with either water or 100 μM GC7 treatment after 48 hours. Immunoblots were stained for hypusine, total eIF5A and β-actin for a control to show even loading. Band intensity graph is shown to the right comparing hypusine to total eIF5A and set relative to water (control). (B) Western blot illustrates protein lysates from 4T1, 67NR and MCF10CA1a cells treated with water or GC7 (0.1→100 μm). Immunoblots were stained for proteins listed in the figure and β-actin as a control. Band intensities are shown directly below for each cell line comparing hypusine to total eIF5A and set relative to water (0 μM). (C) Phase-contrast micrographs were taken 48 hours after water or GC7 treatment before collecting protein lysates for all three cell lines. Time-course of experiment is indicated below panel C.

FIGS. 4A-4C. TGFβ Induces eIF5A Hypusination and Reverses EMT in a Time- and Cell Line-Dependent Manner (A) Western blot illustrates protein lysates treated with BSA (0.1%) or TGFβ (2.5 ng/mL) for 48 hours and immunoblotted for E-cadherin and β-actin as a loading control. Phase-contrast micrographs are shown to the right before collecting protein lysates for all three cell lines. Band intensity graph is shown to the right comparing E-cadherin to β-actin and set relative to BSA (Control). (B) Western blot illustrates protein lysates treated with BSA (0.1%) or TGFβ (2.5 ng/mL) for 10, 30 or 120 minutes and immunoblotted for indicated proteins. Band intensity graph is shown to the right comparing hypusine to total eIF5A for TGFβ treated samples versus control and all set relative to the 10-minute time point. (C) qPCR graphs show relative ZEB1 mRNA expression in 4T1 and MCF10CA1a cells after 1 hour pre-treatment of GC7 (10 μM) and either 12- or 48-hours of TGFβ treatment (2.5 ng/mL) on either plastic or fibronectin (5 μg/mL). Phase-contrast micrographs are shown for all treatments below qPCR graphs collected before RNA extraction at either 12- or 48-hours (Control=BSA/water). Time-course of experiments are illustrated to the right of each panel (Scale bar: 100 μm).

FIGS. 5A-5E. GC7 and Tubastatin A Work Synergistically in 4T1 Cells to Decrease Protein Translation and Block Nuclear Export of eIF5A. Cytotoxicity graphs show cell viability using Aqueous One reagent. Graphs are calculated as percent control. (A) 4T1, 67NR and MCF10CA1a cells were pre-treated with water or GC7 (1 μM). After 12 hours, cells were then treated with DMSO or Tubastatin (0.1 nM→100 μM). After 72 hours, cell viability was quantified. (B) 4T1, 67NR and MCF10CA1a cells were pre-treated with DMSO or Tubastatin A (1 μM). After 12 hours, cells were then treated with water or GC7 (0.1 nM→1 mM). After 72 hours, cell viability was quantified. Time-course of experiments are illustrated to the right of each panel. (C) MTS assay using Aqueous One reagent was used when 4T1 cells were plated and treated with Tubastatin A (TubA) at 10 μM or GC7 at 10 μM or both for indicated time points. Time-course of experiment is illustrated below this panel. * indicates t-test derived p-value less than 0.05. (D) 4T1 cells were either treated with DMSO/water as a control, Tubastatin A (TubA) at 10 μM, GC7 at 10 μM or both Tubastatin A and GC7 at 10 μM for 48 hours before performing immunofluorescence and staining for total eIF5A and DAPI. Phase-contrast and merge channels are also shown (Scale bar: 100 μm). Quantified data is shown to the right of panel D indicated a percent of eIF5A+ nuclei per every spread cell. (E) Total protein is shown by Ponceau S stain for indicated treatments at 10 μM after 48 hours before protein collection. Time-course for experiments displayed in panel D and E are illustrated below panel E.

FIGS. 6A-6D. HDAC6 and DHPS Inhibition Blocks TGFβ-Induced EMT in 4T1 Cells 4T1 cells were plated and treated with either Tubastatin A (TubA) at 10 μM or GC7 at 10 μM or both drugs together. After 48 hours, the media was changed and re-treated with the same drugs at those same concentrations; however, this time with the addition of TGFβ at 2.5 ng/mL. 48 hours after those treatments, RNA was extracted to perform qPCR for ZEB1 expression. (B) The same experiment described in panel A was performed and protein lysates were collected at the 48-hour time point post TubA and GC7 treatment, 24 hours post TubA and GC7 re-treatment and TGFβ treatment labeled as 72 hours as well as 48 hours post TubA and GC7 re-treatment and TGFβ treatment labeled as 96 hours. Ponceau S stain is shown for each time point with respective Western blots shown below. Immunoblotting was stained for E-cadherin, hypusinated eIF5A, total eIF5A and β-actin as a loading control (C) Phase-contrast images of images of 4T1 cells were taken before protein lysate collection described in panel B (Scale bar: 100 μm). (D) 4T1 cells were prepared the same way as described in panel A at the 96-hour time point and stained by immunofluorescence for the indicated proteins, total-eIF5A in green and DAPI to stain the nuclei in blue. Merge channels are also shown as reference. Time-course of all experiments is illustrated to the right of panel A (Scale bar: 100 μm).

FIG. 7. The Role of HDAC6 and DHPS in TGFβ-Induced EMT in Breast Cancer When TGFβ binds to TβRII, HDAC6 is activated to de-acetylate eIF5A in the nucleus. Once de-acetylated, eIF5A is readily available for immediate hypusination and export out into the cytoplasm. There are many proteins that are shuttled out with hypusinated-eIF5A such as Smad3. Once Hyp-eIF5A is in the cytoplasm, PEAK1 gets translated and can promote Src activity in the presence of fibronectin downstream ITGβ3. Src phosphorylates TβRII recruiting Grb2. Following the recruitment of Grb2, PEAK1 promotes the phosphorylation of SMAD2/3 and MAPK. These pathways result in the transcription of genes that promote EMT, cancer cell proliferation and survival. The inventors have demonstrated that by blocking eIF5A deacetylation by HDAC6 using Tubastatin A or SIRTUIN2 using Sirreal2 and inhibiting DHPS by using GC7, one can down-regulate PEAK1 protein, suppress TGFβ-induced EMT and promote nuclear accumulation of eIF5A. There are two alternative drugs available to target HDAC6, Tubacin and Ricolinostat (which is currently in clinical trials).

FIGS. 8A-8E. DHPS/SOX2/TP53 Signaling Axis Associates with Diminished Patient Survival (A) Schematic representing the bioinformatics work flow for identifying eIF5A-PEAK1 EMT (EPE) gene list (implementing array data from Croucher et al.⁵), the resulting Cytoscape Interactome (B), Kaplan-Meyer Survival graphs (C, D), and immunohistochemical (IHC) stains on patient tumor tissues (E). (B) Cytoscape interactome using the EPE gene set with query genes highlighted in yellow. (C, D) Survival of breast cancer patients with and without alterations in the listed genes (SOX2 is co-amplified and TP53 mutated with EPE genes in the Cytoscape interactome) obtained from the METABRIC dataset available on cBioPortal. (E) IHC stains against the indicated proteins in breast tumor samples of patient #1910 obtained from the Human Protein Atlas Pathology Atlas (Scale bars: 100 μm; inset, 50 μm).

FIGS. 9A-9B. (A) IHC patient data from The Human Protein Atlas show proteins from a breast cancer patient exhibiting a mesenchymal phenotype (Scale bar: 100 μm and 50 μm for full image and inlay respectively). (B) Indicated mouse and human breast cancer cells were plated/treated on tissue culture plastic or fibronectin with indicated doses of GC7. Viable cell number was determined by MTS assay analysis.

FIGS. 10A-10C. (10A and B) Immunofluorescence for total eIF5A and DAPI and phase-contract imaging of 67NR (A) and CA1a (B) following 72 hr treatment with Vehicle Control, GC7 (10 uM), TubA (10 uM) or both GC7 and TubA (10 uM ea.). (C) Immunofluorescence for total eIF5A and phase-contrast imaging in 4T1 cells following 24 or 72 hr treatment with Vehicle Control, GC7 (10 uM), TubA (10 uM) or both GC7 and TubA (10 uM ea.).

FIGS. 11A and 11B. Inhibition of DHPS with low doses of GC7 reduces metastasis, while dual inhibition of DHPS/HDAC6 with intermediate doses of GC7/TubA reduces primary tumor growth. Human triple negative MCF10CA1h breast cancer cells were pre-treated with TGFβ and fibronectin for 7 days to induce PEAK1-dependent epithelial-mesenchymal transition and tumorigenic potential. 1e6 cells were xenografted onto the chorioallantoic membrane of 10 day old chicken embryos in 20 uL of growth factor reduced Matrigel, and subsequently treated with a vehicle control, GC7, TubastatinA (TubA) or both GC7/TubA at either 1 uM (panel A) or 10 uM (panel B). Tumors were allowed to develop over the following 7 days and then harvested and weighed along with the indicated liver, lung and brain tissues. gDNA was isolated from these additional tissues to quantify human Alu repeat sequence composition normalized to host chicken GAPDH as a measure of relative cell metastasis to the indicated tissues.

FIG. 12A-12B. GC7-Mediated Hypusination Inhibition Does Not Generally Block Nuclear Export of eIF5A in Breast Cancer Cells (N=2). (A) 4T1, 67NR, BT549, MDA-MB-468, MCF10CA1a, and MCF10CA1h cells were treated with water control, 1 μM GC7, or 10 μM GC7 for 48 hours before immunofluorescent staining against total eIF5A protein and nuclear counterstain using DAPI. Phase-contrast (PhC) images for all cells were obtained prior to staining. Also shown are merged images of total eIF5A and DAPI stains. (B) For each line, cells were assessed manually for depletion of total eIF5A signal strength in nuclei compared to cytoplasm. Error bars represent mean±standard deviation (Scale bar: 100 μm).

### DETAILED DESCRIPTION

Unless defined otherwise, all technical and scientific terms used herein have the same meaning as is commonly understood by one of ordinary skill in the art.

As used herein, treating/treatment means any manner in which one or more of the symptoms of a disease or disorder are ameliorated or otherwise beneficially altered. Treatment also encompasses any pharmaceutical use of the compositions herein, such as use for treating a metabolic disease.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, the term “subject” refers to any animal (e.g., a mammal), including, but not limited to, humans, non-human primates, rodents, and the like, which is to be the recipient of a particular treatment. Preferably the subject is a human.

A method of treating a subject with cancer is contemplated and disclosed herein. Contemplated methods include administering to a subject in need thereof an effective amount of a pharmaceutical composition that includes inhibitors of HDAC6 and/or sirtuins and an inhibitor of deoxyhypusine synthase (DHPS). Methods also include treating a subject with cancer by administering to a subject in need thereof an effective amount of a pharmaceutical composition comprising at least one inhibitor of sirtuin and at least one inhibitor of histone deacetylase six (HDAC6); at least one inhibitor of sirtuin or at least one inhibitor of histone deacetylase six (HDAC6), or a combination thereof. Additional methods include treating a subject with cancer by administering to a subject in need thereof an effective amount of a pharmaceutical composition comprising at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof. As contemplated herein, cancer may include any type of cancer, including breast cancer or pancreatic cancer.

A contemplated pharmaceutical treatment for treating a subject with cancer comprises at least one inhibitor of sirtuin and at least one inhibitor of histone deacetylase six (HDAC6); at least one inhibitor of sirtuin or at least one inhibitor of histone deacetylase six (HDAC6), or a combination thereof.

Another contemplated pharmaceutical treatment for treating a subject with cancer comprises at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof.

A contemplated composition is contemplated that comprises at least one inhibitor of sirtuin and at least one inhibitor of histone deacetylase six (HDAC6); at least one inhibitor of sirtuin or at least one inhibitor of histone deacetylase six (HDAC6), or a combination thereof.

Another contemplated composition is contemplated that comprises at least one inhibitor of sirtuin, an inhibitor of deoxyhypusine synthase (DHPS), and at least one inhibitor of hi stone deacetylase six (HDAC6); at least one inhibitor of sirtuin, an inhibitor of deoxyhypusine synthase (DHPS), or at least one inhibitor of histone deacetylase six (HDAC6), or a combination thereof.

Formulation of Pharmaceutical Compositions

The pharmaceutical compositions provided herein contain therapeutically effective amounts of one or more of compounds provided herein in a pharmaceutically acceptable carrier.

The compositions contain one or more compounds provided herein. The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation, creams, ointments and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of conditions including, but not limited to, undesired cell proliferation, cardiovascular, renal, neurodegenerative/neurologic and ophthalmic disorders, diseases or syndromes characterized by chronic inflammation, cardiovascular diseases and cancers as described herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated undesired cell proliferation, cardiovascular, renal, neurodegenerative/neurologic and ophthalmic disorders, diseases or syndromes characterized by chronic inflammation, cardiovascular diseases and cancers as described herein.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of compound per kilogram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases or disorders associated with undesired cell proliferation, cardiovascular, renal, neurodegenerative/neurologic and ophthalmic disorders, diseases or syndromes characterized by chronic inflammation, cardiovascular diseases and cancers as described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of administration. Preferred modes of administration include parenteral and oral modes of administration. Oral administration is presently most preferred.

Solutions or suspensions used for parenteral, intradermal, subcutaneous, or topical application can include any of the following components: a sterile diluent, such as water for injection, saline solution, polysorbate (TWEEN 80), fixed oil, polyethylene glycol, glycerine, propylene glycol or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses, which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases or disorders associated with undesired cell proliferation, coronary restenosis, osteoporosis, syndromes characterized by chronic inflammation, autoimmune diseases and cardiovascular diseases. It is to be understood that such combination therapy constitutes a further aspect of the compositions and methods of treatment provided herein.

Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic□coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Injectables, Solutions and Emulsions

Parenteral administration, generally characterized by injection, either subcutaneously, intrathecal, intrathecal, epidural, intramuscularly or intravenously is also contemplated herein. Injectables can be prepared in conventional forms, either as liquid solutions or suspensions; solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

It is generally accepted that epithelial-mesenchymal transition (EMT) is an important component of the metastatic cascade in solid tumor types such as breast cancer. In this regard, the inventors have previously established that PEAK1 promotes breast cancer metastasis by switching Transforming Growth Factor β (TGFβ) signaling toward its EMT-promoting functions. Eukaryotic Initiation Factor 5A 1/2 (eIF5A1/2) are unique translation factors in that they are the only known protein substrates for the post-translational hypusine modification—a key modification required for eIF5A translation activity. Since eIF5A is required for Pseudopodium-Enriched Atypical Kinases 1 (PEAK1) translation, the inventors hypothesized that TGFβ may induce PEAK1 upregulation during EMT by directly activating the eIF5A hypusination pathway. Evidence of an active eIF5A/PEAK1 pathway in undifferentiated, mesenchymal breast cancer tissue is provided. Notably, inhibition of eIF5A hypusination blocks PEAK1 translation, cell viability and TGFβ-induced EMT in breast cancer cells. In this regard, the inventors demonstrate that TGFβ induces post-translational hypusination/activation of eIF5A in metastatic breast cancer cells.

TGFβ is known to activate other eIF5A regulatory enzymes that have previously been reported to mediate EMT in breast cancer. For example, TGFβ-induced EMT requires Activin Receptor Type-1B (ACVR1B/ALK4)-dependent Histone Deacetylase 6 (HDAC6) activation and HDAC6 promotes eIF5A deacetylation leading to its rapid nuclear export and hypusination. The inventors hypothesized that cytoplasmic localization of eIF5A and eIF5A hypusination are required for cell proliferation/survival and TGFβ-induced EMT in breast cancer. Since HDAC inhibitors are promising new anti-cancer agents being evaluated in clinical trials, the inventors designed experiments to test whether blockade of eIF5A hypusination could increase the potency or efficacy of HDAC6 inhibitors. Most notably, the inventors demonstrate that dual treatment with non-cytotoxic doses of HDAC6 and eIF5A hypusination inhibitors synergize to potently and selectively kill metastatic breast cancer cells and block TGFβ-induced EMT. This also resulted in a further accumulation of eIF5A in the nucleus regardless of TGFβ treatment. In this regard, the inventors have formulated a pathway in which it is believed that TGFβ stimulates HDAC6 and DHPS function to export eIF5A into the cytoplasm and promotes PEAK1 translation to result in EMT, invasion and metastasis in breast cancer cells.

Contemplated subject matter demonstrates that during blockade of hypusination activity using GC7 (a specific DHPS inhibitor), both cell number and PEAK1 protein are downregulated 48 hours post treatment. It was shown that these effects are dose-dependent in a metastatic mouse breast cancer cell line. Induction of EMT by TGFβ treatment increases GC7 potency complementing the reverse discovery that inhibition of eIF5A hypusination blocks TGFβ-induced EMT. During simultaneous HDAC6 and DHPS inhibition, the inventors identified a decrease in cell number, ZEB1 mRNA expression and a significant nuclear accumulation of eIF5A, which are all signs of reduced EMT/metastasis.

### EXAMPLE 1

eIF5A Isoform Expression Levels Predict Hypusination Profile in Breast Cancer Lines

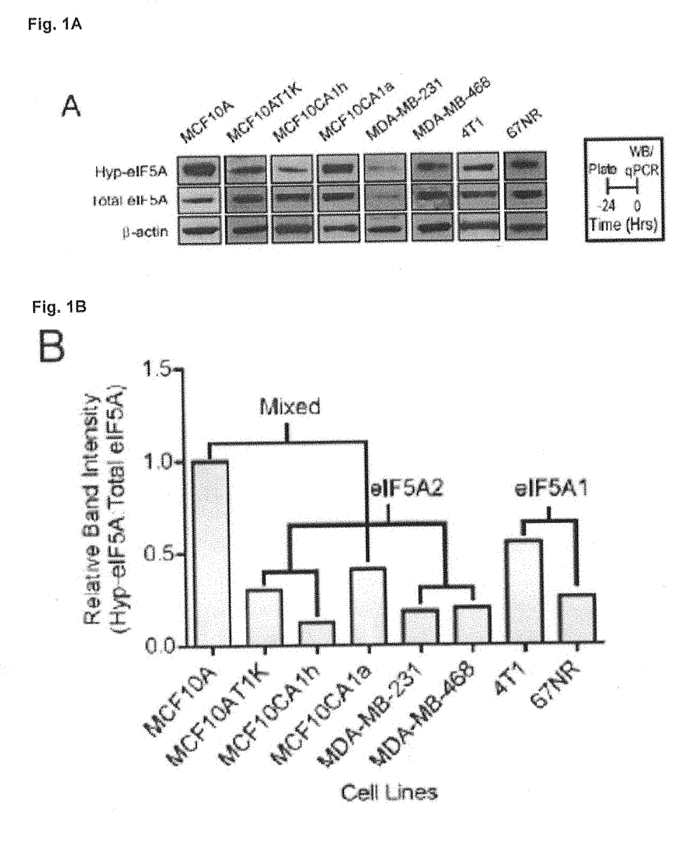

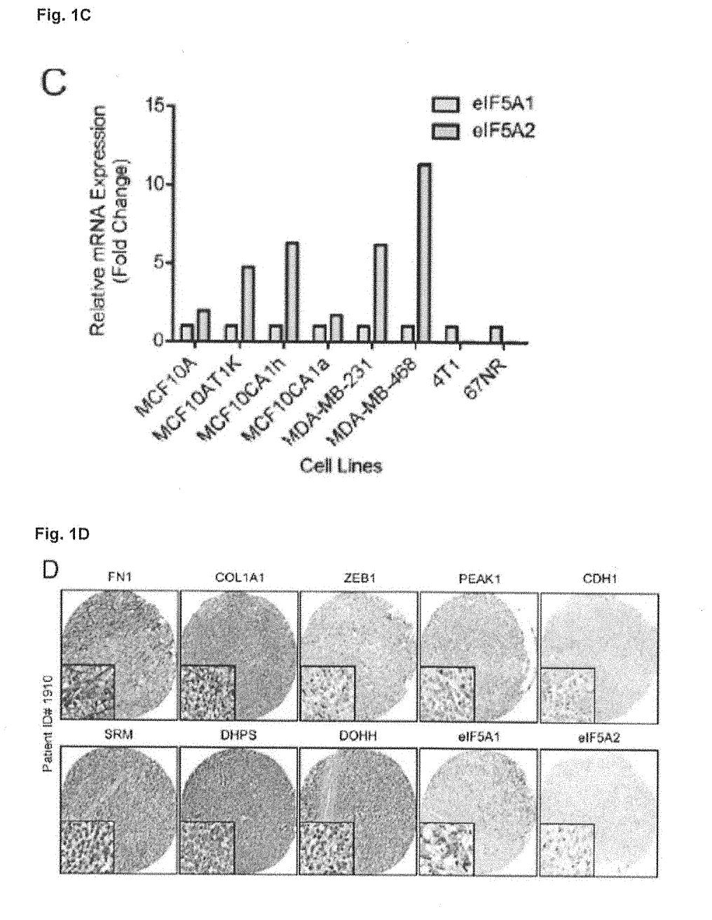

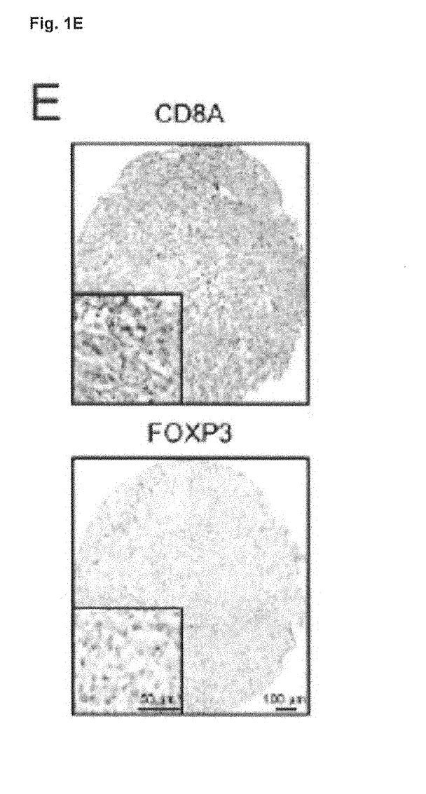

To determine if eIF5A is expressed in multiple cancer cell types, basal levels of hypusine and total eIF5A in a range of cell lines that represent breast cancer were analyzed. To do this, following cell lines were used: MCF10A—a nonmalignant human breast cell line, along with three HRas transformed derivatives of MCF10A cells, MCF10AT1k, MCF10CA1h, MCF10CA1a. Two human triple negative breast cancer (TNBC) cell lines, MDA-MB-231 and MDA-MB-468, as well as two mouse malignant breast cancer cell lines 4T1 and 67NR which are considered to be of a TNBC subtype were also used. These cells were plated and left to attach overnight. The protein lysates were then collected and a western blot was performed using antibodies against hypusine, total eIF5A and β-actin as a loading control (FIG. 1A). To quantify this western blot, a relative band intensity graph is shown in FIG. 1B. The graph discloses the ratio between hypusine to total eIF5A for each cell line. All band ratios are set relative to the non-malignant human mammary cell line, MCF10A. The bars on top of the graph represent a relationship describing which eIF5A isoform is predominantly being hypusinated based on qPCR results in FIG. 1C. To identify the eIF5A isoform levels, RNA was extracted from the cell lines and the RNA was reverse transcribed to cDNA before performing a qPCR experiment. To quantitate the levels of eIF5A isoforms, primers against each isoform were used independently and the levels of isoform 2 were set relative to isoform 1 (FIG. 1C). Here, it is seen that in all cell lines represented except 4T1 and 67NR cells, the predominantly expressed isoform is eIF5A1. Thus it is assumed that most of the hypusinated eIF5A shown in the western blot for 4T1 and 67NR is due to eIF5A isoform 1. MCF10A and its HRas transformed derivative, MCF10CA1a seem to have similar levels of isoforms 1 and 2; therefore, it was concluded that both isoforms are responsible for hypusination activity for those two cell lines. The remaining four cell lines, both HRas derivative of MCF10A, MCF10AT1k and MCF10CA1h as well as the two TNBC cell lines, 4T1 and 67NR showed much higher levels of eIF5A2, so it was concluded that the hypusination activity in those cells was due to eIF5A isoform 2 (FIG. 1B). Data from the Human Protein Atlas shows evidence of this pathway being expressed (FIG. 1D). In FIG. 1D, IHC staining on tissue samples from a breast cancer patient exhibiting a mesenchymal phenotype was performed to indicate the levels of mesenchymal markers such as fibronectin (FN1), COL1A1 and ZEB1. All mesenchymal markers are expressed at elevated levels, while the epithelial marker, CDH1 (E-cadherin) is expressed at low levels. PEAK1 staining matches that of mesenchymal markers as well as the pathway in which it is translated by the staining of spermidine synthase (SRM), Deoxyhypusine synthase (DHPS), Deoxyhypysine hydroxylase (DOHH) and the two isoforms of eIF5A, 1 and 2 (FIG. 1D). A study done by Imam's group demonstrated that eIF5A positively regulates the recruitment of CD4+/CD8+ T cells to the site of the tumor. Imam's group research this relationship in pancreatic cancer cells in which during GC7 treatment to block hypusination/activation of eIF5A, recruitment of CD4+ T cells significantly decrease at the site of the tumor; however, pancreatic and splenic CD8+ lymphocytes did not seem to be affected²⁸. The group also reported that a specific transcription factor, FOXP3+, found in regulatory T cells in significantly increased to the site of the tumor during GC7 treatment²⁸. This suggests an inverse relationship between CD8+ T cells and FOXP3+ regulatory T cells. To validate this in breast cancer tissue, IHC staining for CD8A (CD8+ T cells) and FOXP3 (FOXP3+ regulatory T cells) are shown in breast cancer tissue samples taken from The Human Protein Atlas also correlating with high PEAK1 levels (FIG. 1E). Based on these data, it was concluded that the eIF5A isoform expression levels present in the breast cancer tissue can predict hypusination profiles and lymphocyte microenvironment of the primary tumor.

### EXAMPLE 2

GC7 Decreases Cell Proliferation/Number Across Breast Cancer Cell Lines

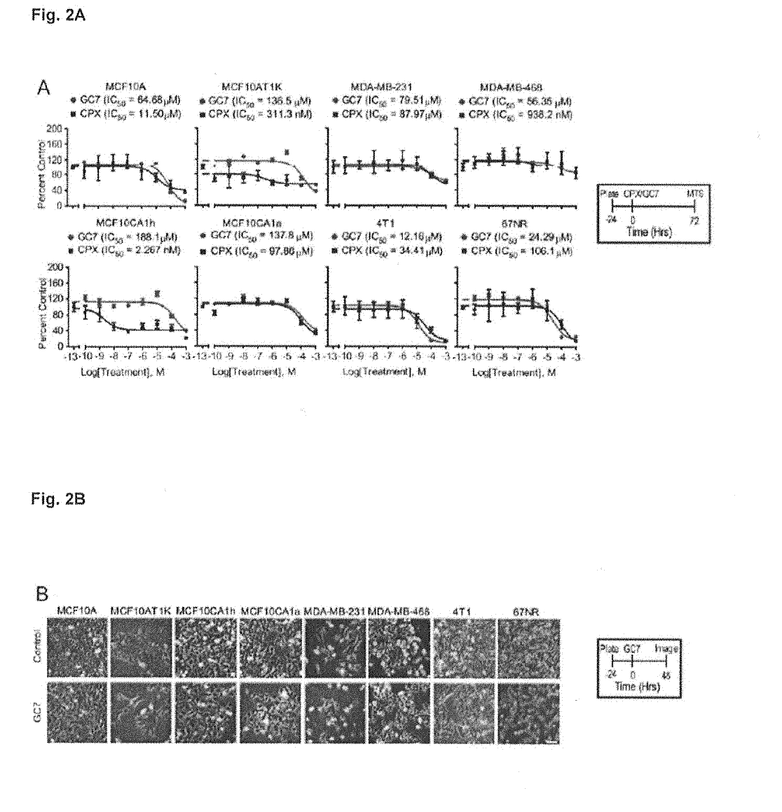

There are currently two drugs available that can target the activation of eIF5A. Its role of hypusination/activation is spermidine dependent and is carried out in two subsequent steps by the enzymes deoxyhypusine synthase (DHPS) catalyzing the first step and deoxyhypusine hydroxylase (DOHH) catalyzing the second step. The efficacy and potency of the two drugs in the selected cell lines; GC7 (N1-Guanyl-1,7-diaminoheptane)—a selective inhibitor of DHPS, and CPX (Ciclopirox olomine)—which chelates iron that is important for the activity of DOHH (FIG. 2A) are shown. The concentration that determines a 50% decrease in cell viability is noted by the IC₅₀ values on each graph for each drug. These cells were imaged when treated with the second highest concentration of GC7 at 100 μM to show a decrease in cell viability after 48 hours of treatment (FIG. 2B). In conclusion, it was demonstrated that CPX has numerous amounts of off-target effects—such as chelating iron as well as inhibiting cell survival and other necessary metabolic pathways, suggesting that GC7 was a more specific drug to use to target the eIF5A pathway. With that in mind, the inventors moved forward with selected cell lines 4T1 and 67NR which exhibiting the largest efficacy after GC7 treatment and MCF10CA1a which is a highly aggressive form of the HRas transformed MCF10A cell line. From this point forward, all experiments performed used only GC7 as the selected drug to target hypusinated eIF5A.

### EXAMPLE 3

GC7 Inhibits eIF5A Hypusination and Cell Number in a Dose-Dependent Manner

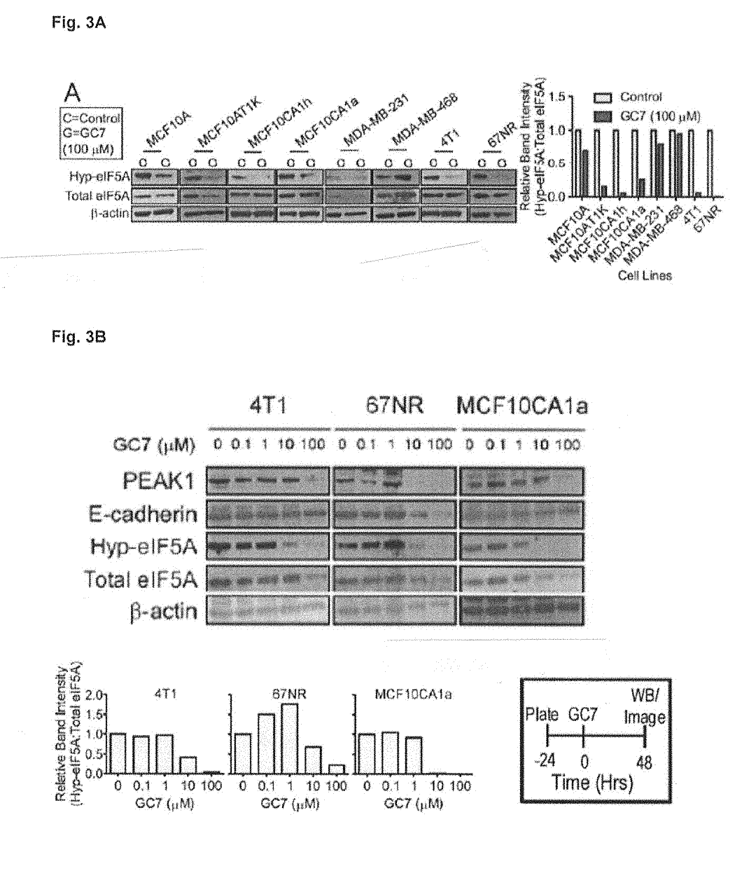

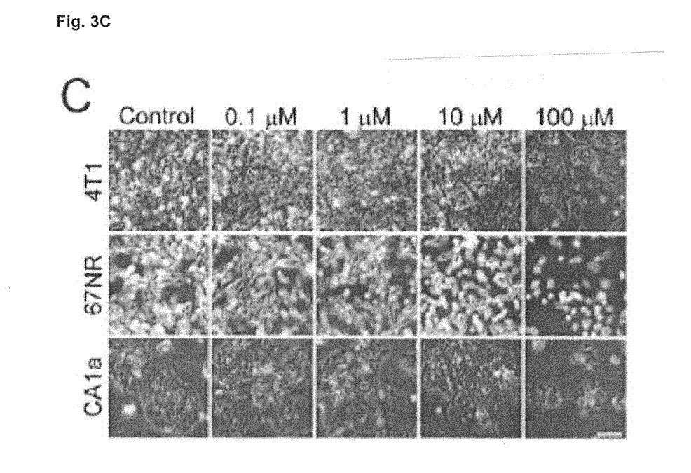

The inventors sought to determine if the decrease in cell viability is due to the inhibition of post translational modification of eIF5A. To test this, all cell lines were treated with GC7 at 100 μM for 48 hours—which were the time point and concentration at which a decrease in cell viability was seen (FIG. 2B). The inventors then collected, performed a western blot and immunoblotted for hypusine, total eIF5A and β-actin as a loading control (FIG. 3A). The cell lines with the largest decrease in cell viability at 100 μM GC7, also had the largest decrease in hypusinated eIF5A protein activity (4T1, 67NR, MCF10AT1k, MCF10CA1h and MCF10CA1a). To test if this effect is dose-dependent, 4T1, 67NR and MCF10CA1a cells were treated with various concentrations of GC7 ranging from 0.1 μM to 100 μM. Protein lysates were then collected after 48 hours of treatment and performed a western blot. A previously published paper shows that when eIF5A is in its active form, translation of PEAK1 occurs pancreatic cancer cells¹³. To validate this in breast cancer cells, the inventors immunoblotted for PEAK1 as well as E-cadherin, hypusine, total eIF5A and β-actin as a loading control (FIG. 3B). As seen in the results of FIG. 3B, there is a striking down regulation of PEAK1 protein in a dose-dependent manner in 4T1, 67NR and MCF10CA1a cells. In contrast, during the decrease in PEAK1 expression, a decrease in E-cadherin—an epithelial marker with increased GC7 concentration only in the 67NR cell line—was identified. Hypusinated eIF5A levels consistently decreased (shown by band intensity ratios between total eIF5A and hypusine below each respective western blot) (FIG. 3B). The inventors next looked at cell viability at lower concentrations of GC7, such as 1 μM and 10 μM, where a decrease in hypusinated eIF5A is still achieved but the decrease in cell viability is not as great in lower concentrations that do still target hypusinated eIF5A (FIG. 3C). Taken together, hypusination activity and PEAK1 expression can successfully be downregulated using GC7 dose- and cell line-dependent manner. In this regard, using lower doses to achieve the same affect can be demonstrated without additive decrease in cell number.

### EXAMPLE 4

TGFB Induces eIF5A Hypusination and Reverses EMT in a Time- and Cell Line-Dependent Manner

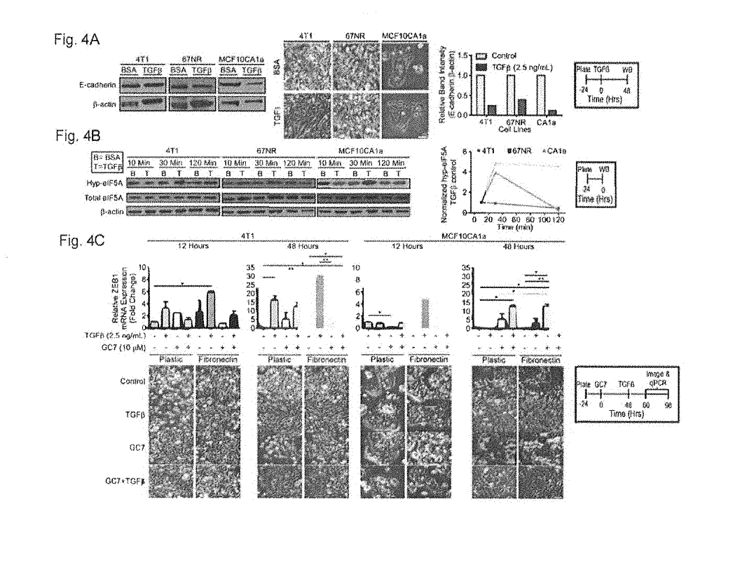

The pathway in which the inventors are interested in targeting eIF5A is in the context of TGFβ-induced EMT and TGFβ is a well-characterized inducer of EMT. The inventors sought to demonstrate a TGFβ-induced EMT effect in 4T1, 67NR and MCF10CA1a cells by performing a western blot using antibodies for E-cadherin—an epithelial marker. Phase-contrast images are shown to the right of the western blot to indicate a shift in morphology after treatment with TGFβ. Relative band intensity is graphed to its right to show a true decrease in E-cadherin expression (FIG. 4A). Since TGFβ can induce PEAK1 upregulation, while in contrast, hypusinated eIF5A promotes PEAK1 translation, the inventors sought to see if TGFβ alone can upregulate hypusinated eIF5A. Post-translational modifications can occur relatively fast. Therefore short-term treatments of TGFβ on 4T1, 67NR and MCF10CA1a cells were performed initially. Protein lysates were collected after 10, 30 and 120 minutes of treatment and immunoblotted for hypusine, total eIF5A and β-actin (FIG. 4B). The inventors demonstrated that TGFβ has the ability to activate a post-translational modification. Relative band intensity is graphed to the right of the western blot in which the ratio between hypusine to total eIF5A is compared first between treated samples to control samples then a ratio is calculated comparing each time point relative to 10 minutes as a starting time point of reference. This data shows a true increase in hypusinated eIF5A in 4T1 and MCF10CA1a cells by 30 minutes of TGFβ treatment; however, the increase is maintained through 120 minutes of TGFβ treatment in 4T1 cells, but not in MCF10CA1a cells (FIG. 4B). The inventors were also interested in what genes were altered at the transcriptional level when dual treating with TGFβ and GC7 at the same time to block EMT. To test for this, 4T1 and MCF10CA1a cells were plated on either plastic or fibronectin extra-cellular matrix (ECM) protein and pre-treated them with GC7 at 10 μM for 48 hours to ensure the eIF5A hypusination pathway is blocked. The inventors then treated with TGFβ at 2.5 ng/mL and collected RNA after 12 or 48 hours. Interestingly, after performing qPCR for a mesenchymal marker, ZEB1 expression was decreased when treating with both GC7 and TGFβ after 12 hours on plastic; however, this effect was blocked on fibronectin in 4T1 cells. This effect was not seen after 48 hours of treatment in 4T1 cells (FIG. 4C). To be able to induce EMT in MCF10CA1a cells by treating with TGFβ was possible only on fibronectin; however, this EMT was blocked with dual treatment of GC7 and TGFβ after 48 hours (FIG. 4C). Phase contrast images were also taken before RNA collection to show changes in morphology where EMT was either induced or reversed (FIG. 4C). Based on these data, it was concluded that although TGFβ is a well-characterized inducer of EMT, it can also promote the post-translation modification of eIF5A hypusination. Also, with the correct treatment time and conditions, inhibition of hypusination can block TGFβ-induced EMT.

### EXAMPLE 5

GC7 and Tubastatin A Work Synergistically in 4T1 Cells to Decrease Protein Translation and Block Nuclear Export of eIF5A

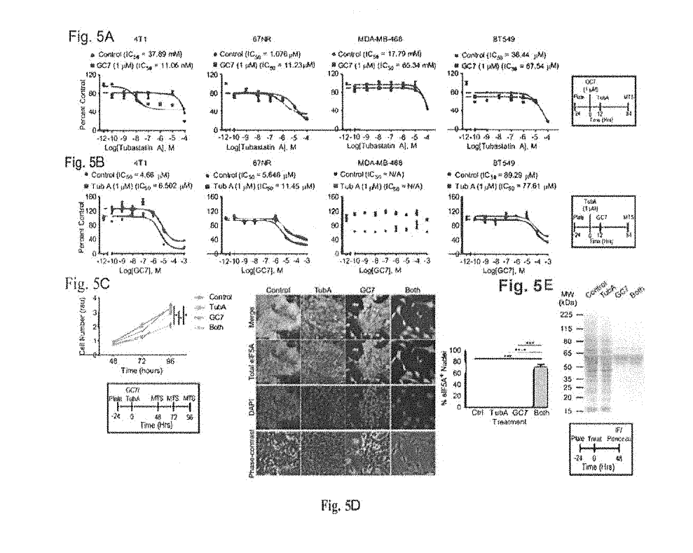

The next research questions revolved around asking how TGFβ is regulating eIF5A and what other proteins might be involved to completely block TGFβ-induced EMT. This led to histone deacetylase enzyme 6 (HDAC6). HDAC6 has been previously reported to be responsible for the deacetylation of eIF5A in the nucleus in order to make it readily available for hypusination and later transport out in the cytoplasm for functioning as a translation factor. To determine if it were possible to further increase the efficacy or potency of GC7, the inventors co-treated with Tubastatin A (an HDAC6 inhibitor) in 4T1 and 67NR cells as well as MDA-MB-468 cells which initially were not very sensitive to GC7. Triple negative human breast cancer cell line, BT547, was also used to see if another cell line representative of this breast cancer subtype was sensitive towards DHPS and HDAC6 inhibition. (FIGS. 5A and 5B). Here, are shown cell viability graphs for these four cell lines pretreated with GC7 at 1 μM and after 12 hours were exposed to various concentrations of Tubastatin A (TubA) ranging from 0.1 nM to 100 μM. Interestingly, GC7 treatment dramatically increased the potency of TubA in the metastatic mouse mammary cell line, 4T1; however, this effect was not seen in the non-metastatic mouse mammary counter-part, 67NR. MDA-MB-468 and BT549 cells remained to be insensitive to TubA with or without GC7 pretreatment (FIG. 5A). Next, the inventors performed the opposite cell viability experiment in which these four cell lines were pretreated with TubA at 1 μM and then exposed these cells to various concentrations of GC7 ranging from 0.1 nM to 1 mM. In this instance, TubA treatment subtly decreased the potency of GC7 in the metastatic mouse mammary cell line, 4T1; however, this effect was not seen in the three other cell lines used (FIG. 5B). In order to determine if the drugs are working synergistically, the inventors again performed the cell viability assay using the 4T1 cells which showed an increase in TubA drug potency when pretreating with GC7. 4T1 cells were treated with TubA at 10 μM, GC7 at 10 μM or both drugs simultaneously for 48, 72 and 96 hours. The results indicate that these drugs work synergistically to decrease cell viability at the 96-hour time point (FIG. 5C). Previously published data revealed that HDAC6 is responsible for the localization of eIF5A²¹. To test this, the inventors looked further into the changes of eIF5A localization when treating with TubA, GC7 or both drugs simultaneously. In FIG. 5D is shown immunofluorescence staining of 4T1 cells treated with TubA at 10 μM, GC7 at 10 μM or both drugs at those same concentrations for 48 hours. In FIG. 5D, total eIF5A in green is predominantly cytoplasmic in all treatments other than the dual treatment where green fluorescence is evenly distributed across the nuclei and the cytoplasm of the cells. DAPI was used to stain the nuclei shown in the red channel and phase-contrast images are shown as reference (FIG. 5D). To quantify these data, the number of spread cells for every image taken for each condition was counted and then calculated the percent of those spread cells with an eIF5A+ nuclei indicated by a green colored nucleus (FIG. 5D). Protein lysates were also collected at the same time point described in FIG. 5D. Twenty μg of protein for each condition was run on a Bis-Tris agarose gel then transferred onto a nitrocellulose membrane before staining total protein using Ponceau S (FIG. 5E). In this regard, it was concluded that GC7 and TubA work synergistically to decrease cell viability, total protein translation and promote nuclear accumulation of eIF5A in a mouse metastatic breast cancer cell line.

### EXAMPLE 6

HDAC6 and DHPS Inhibition Blocks TGFB-Induced EMT in 4T1 Cells

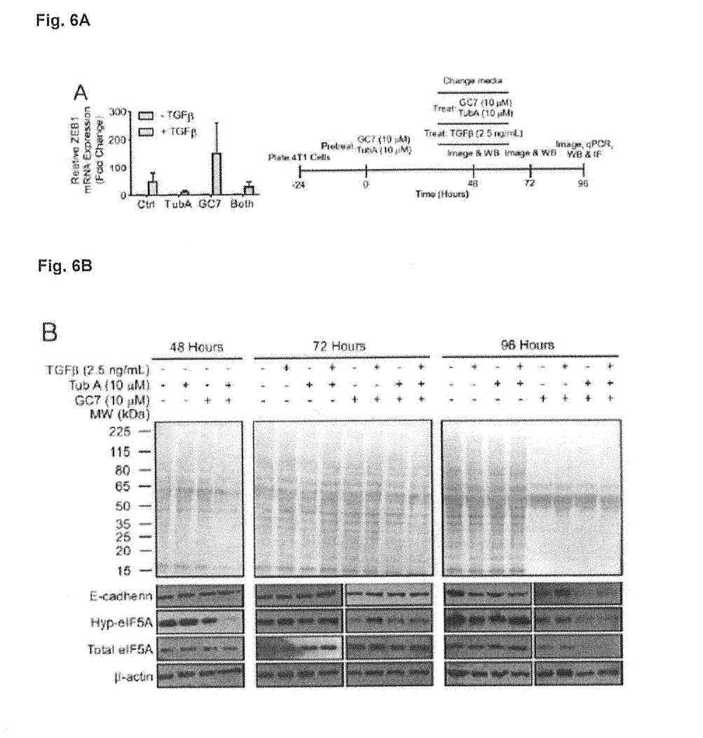

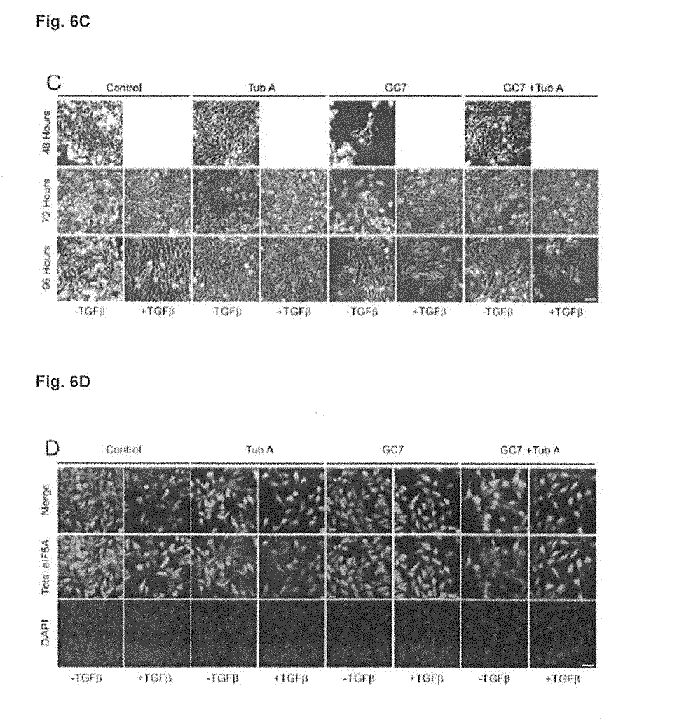

Next, the inventors investigated whether blockade of HDAC6 and DHPS activity could further suppress TGFβ-induced EMT either independently or simultaneously. To test this, 4T1 cells were treated with either TubA at 10 μM, GC7 at 10 μM or both for 48 hours. At this time point, protein lysates were collected as well as phase-contrast images (FIGS. 6B and 6C). At that same time point, the media was changed and the cells were re-treated with the same drugs at the same concentrations; however, this time with the addition of TGFβ at 2.5 ng/mL. The inventors collected protein lysates and phase-contrast images just one day post TGFβ treatment, calling this the 72-hour time point (FIGS. 6B and 6C). In addition, the inventors collected mRNA, protein lysates, phase-contrast and immunofluorescences images two days post TGFβ treatment, calling this the 96-hour time point (FIGS. 6A, 6B, 6C and 6D). In FIG. 6A, qPCR results collected at the 96-hour time point revealed that treatment of TubA blocks TGFβ-induced EMT by decreasing a mesenchymal marker, ZEB1. Also, treatment with GC7 failed to block TGFβ-induced EMT; however, to complement this data, E-cadherin protein expression dramatically increased at this same time point under the same conditions in FIG. 6B. Dual treatment of these drugs suppressed TGFβ-induced EMT shown both in FIGS. 6A and 6B at the transcriptional and translational level. In agreement with the Ponceau S stains of total protein shown in FIG. 6B, FIG. 6C shows a drastic reduction in cell number when treated with GC7 or dual treatment at the 96-hour time point. Also at the 96-hour time point, immunofluorescence images indicate that eIF5A localization is accumulated at the nucleus during TGFβ treatment alone, as seen in TubA treatment with or without treatment of TGFβ treatment (FIG. 6D). Interestingly, dual inhibition independent of TGFβ treatment resulted in accumulation of eIF5A in the nucleus at the 96-hour time point (FIG. 6D). From this data, the inventors conclude that HDAC6 is responsible for the preparation of eIF5A export and DHPS works in synergism with HDAC6 to further promote eIF5A export. If both components are blocked, this results in an accumulation of eIF5A in the nucleus in which TGFβ cannot rescue.

Eukaryotic initiation factors have been implicated in tumor progression and angiogenesis for many cancers. A major hurdle with targeting these proteins is that many translational and biologically processes can be affected downstream of them. The inventors' interests lie in targeting eukaryotic initiation factor 5A (eIF5A) because this specific translation factor carries a unique post-translational modification termed hypusine which makes it an unambiguous target. Once eIF5A is in an active form, its activity in promoting protein synthesis and cell growth are fulfilled. Over the past decade, much research has been performed to study the sequence and molecular characterization of eIF5A and its two isoforms: eIF5A1 and eIF5A2. These two isoforms share about 84% identity and 94% similarity to each other and are about 17 kilodaltons (kDa) in size²⁹. However, some researchers claim that their biological functions may be different. In a study done by Cracchiolo et al. in 2004, it was shown that these two isoforms are ubiquitously expressed in proliferating cells; however, eIF5A2 has alternative expression levels and seems to be cell line specific³⁰. Cracchiolo et al. also propose that eIF5A1 be ranked among the top biomarkers of interest to detect abnormally high proliferative cells found in the intraepithelial neoplasia of the vulva³⁰. There are currently a few reports suggesting the subcellular localization of eIF5A in cells. It was previously suggested that nuclear accumulation of the unmodified form of eIF5A promoted pro-apoptosis, while its hypusinated form could play a role in cell survival³¹.

The inventors began this research with eight cell lines representing various subtypes of breast cancer, including one non-malignant human mammary cell line, MCF10A. Characterization of these cell lines with basal levels of hypusine and total eIF5A permitted the selection of a few cell lines that exhibited elevated levels of hypusination activity (FIG. 1A). Although all cell lines responded to GC7 treatment, human triple negative breast cancer cells remained to be insensitive (FIGS. 2A and 3A). It was also of interest to discover that the non-malignant mammary cell line, MCF10A, was one of the most sensitive to GC7 treatment. This may be because eIF5A has such a critical role in protein synthesis and cell survival during normal functioning, and because the non-tumor cells have been proliferatively immortalized. As seen in pancreatic cancer cell lines shown by Fujimura et al., it was shown that PEAK1 protein expression can be down-regulated by treatment of GC7 in a dose-dependent manner at the 48-hour time point (FIGS. 3B and 3C). Although hypusinated eIF5A protein expression can be decreased at lower concentrations, PEAK1 protein expression is targeted at higher concentrations suggesting a possible proteolytic or translational rescue (FIG. 3B). This suggests a new therapeutic concept to target metastatic triple negative breast cancer (TNBC) cells.

Regarding new concepts to target metastatic TNBC, a review by Hu et al. describes the most common therapies for breast cancer and how their outcomes differ between treating different breast cancer subtypes. The breast cancer stage that remains to be the most difficult to treat and also has the lowest patient survival is stage 4 or the metastatic form of this disease³². A study done in 2013 by Loi et al. showed that TNBC cells have a higher percentage of tumor infiltrating lymphocytes (TIL) present at the site of the primary tumor which also correlates with the drastic decrease in 5-year survival rates when diagnosed with this breast cancer subtype³³. Also, correlating with this data, TIL that are present tend to be more commonly represented as CD8+ T cells which accumulate at the tumor stroma³⁴. Previously published data show that TIL correlate with increasing overexpression of programmed cell death (PD-L1) present on TNBC cells³⁵. PD-L1 overexpression is significantly associated with increased tumor grade, tumor size as well as induced tumor cell proliferation indicated by upregulation of Ki-67 expression³⁶. A study done by Colvin et al. in 2013 discloses that with increasing concentrations of GC7, a significant reduction in FOXP3+ T regulatory cell populations, which have an inverse relationship with CD8+ TIL, were observed in type 1 diabetic mice in vivo³⁷. This suggests a positive regulation between eIF5A expressing TNBC cells and PD-L1 ligand attracting FOXP3+ T regulatory cells. This crosstalk between TNBC cells and the immune system suggests an acquired resistance to the immune engagement regulated by PD-L1/eIF5A expression. In this research, the inventors also present evidence of FOXP3+ T regulatory cells and CD8+ T cells present in a mesenchymal breast cancer patient tissue exhibiting high PEAK1 and hypusination pathway component protein expression (FIGS. 1D and E).

Majority of patient death during cancer progression is due to a metastatic form in which the primary cancer cells have disseminated or invaded into the blood stream, colonizing other distant organs. EMT can be defined as the process in which precedes metastasis³⁸. When cancer cells undergo EMT, they lose their apical to basal polarity, break away from the underlying basement membrane in which they were interacting with, decrease their cell-cell junctions and form a more spindle-like morphology making them invasive, migratory and highly metastatic. In 2009, the Weinberg lab reviewed EMT processes in cancer progression in which the lab proposed a model illustrating the changes between the cell-extra cellular matrix (ECM) interactions leading to a malignant phase of tumor growth. Once this interact is broken, this promotes cancer cells to break away and feed into the bloodstream and form micro- and macro-metastases at remote sites where the cells will then undergo mesenchymal to epithelial transition (MET) to revert to an epithelial phenotype³⁸. One of the most common and well characterized inducers of EMT is TGFβ in which epithelial markers such as Epithelial Cell Adhesion Molecule (EPCAM), Cadherin 1 (CDH1) and Mucin 1 (MUC1) are downregulated and mesenchymal markers such as Zinc Finger E-Box Binding Homeobox 1 (ZEB1) and Cadherin 2 (CDH2) are upregulated³⁹. Many studies have been published showing a direct association between a downregulation of E-cadherin and promotion of EMT. In contrast, a previously published paper described a polarity protein, PAR6, being a key regulatory of tight cell to cell junctions in order to form epithelial cell polarity and plasticity necessary at a metastatic cancer site to form a secondary tumor to undergo MET⁴⁰.

Transforming growth factor beta (TGFβ) is a well-known inducer of epithelial to mesenchymal transition (EMT). The inventors used TGFβ to induce EMT in the selected cell lines that showed the highest efficacy and potency to GC7 treatment. Evidence that TGFβ can induce a translational activity by increasing hypusinated eIF5A protein expression was demonstrated. This induction is a seen briefly at 30 minutes; however, this is maintained for 2 hours in a metastatic mouse cell line (FIG. 4B). In view of this data, the inventors investigated whether a blockade in hypusination could suppress TGFβ-induced EMT. The results revealed a significant decrease in mesenchymal marker mRNA expression levels when pre-treating with GC7. In addition, the inventors tested other factors that could be involved to accomplish a complete blockade of TGFβ-induced EMT (FIG. 4C).

Inhibition of eIF5A Hypusination Reduces Fibronectin- and TGFβ-Dependent Metastatic Dissemination of MCF10CA1h Cell In Vivo

Based on the partially impaired expression of Zeb1 in vitro, we wanted to assess whether this effect would result in altered metastatic dissemination using the in vivo chicken CAM system. We had previously used this system to demonstrate that metastatic spread but not primary tumor growth of MCF10CA1h cells is dependent on PEAK1 expression under fibronectin- and TGFβ treatment conditions¹⁰. Using identical treatment conditions, we observed that treatment of xenografted MCF10CA1h cells with 1 μM GC7 resulted in significantly reduced metastatic dissemination to lung but not liver tissues, while primary tumor mass was unaffected.

Subcellular Localization of eIF5A is Highly Restricted in MCF10CA1h Cells

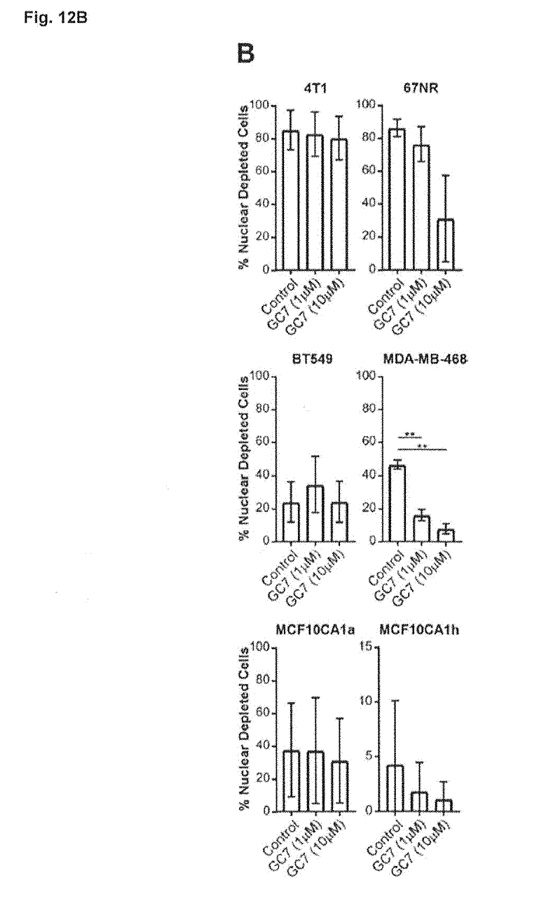

Since hypusination of eIF5A has been associated with significantly increased affinity for nuclear export via exportin-4⁷, and thus as a requisite for translational activity of eIF5A, we wanted to assess whether the cell survival and metastatic effects we observed were linked to subcellular localization patterns of eIF5A proteins. Thus, we interrogated the subcellular localization of eIF5A protein using immunofluorescent staining under control as well as 1 and 10 μM GC7 treatment conditions in triple negative breast cancer lines (FIGS. 12A and 12B). This analysis revealed that different cell lines maintain a wide range of subcellular localizations of eIF5A protein under control conditions, with 4T1 and 67NR cells showing little nuclear signal. Notably, MCF10CA1h cells exhibited the greatest proportion of nuclear staining under control conditions, thus suggesting that in these cells eIF5A protein is primarily restricted to the nucleus (FIG. 12A). Nuclear depletion of eIF5A was observed in 67NR, MDA-MB-468, and MCF10CA1h cells following GC7 treatment, though this change was significant only in the case of MDA-MB-468 cells (FIG. 12B). MCF10CA1a cells exhibited high inter-cellular variability, with some cells clearly displaying nuclear enrichment of eIF5A protein while other cells showed nuclear depletion (FIG. 12A).

In this research, a treatment in which TGFβ-induced EMT can be suppressed is proposed. Since HDAC6 inhibition results in nuclear accumulation of eIF5A also reducing translational activity, the inventors used Tubastatin A (TubA) to target HDAC6. During dual inhibition of HDAC6 and DHPS, it was found that these drugs work synergistically to decrease cell number, protein translation as well as nuclear accumulation of eIF5A (FIGS. 5A, 5B, 5C and 5D). This finding suggests a condition which may cause cancer cells to be sensitive to chemotherapy and drug treatments. By manipulating a cancer cell to become apoptotic by nature could reverse chemo-resistance, specifically metastatic TNBC cells and increase a vulnerable state to increase patient survival. To this extent, the inventors sought to see if TGFβ-induced EMT could further be blocked during dual inhibition. In the research described above, pre-treatment with a HDAC6 inhibitor and a DHPS inhibitor significantly decreased TGFβ-induced EMT shown by qPCR and western blot (FIGS. 6A, 6B and 6C). During DHPS inhibition, TGFβ-induced EMT was reversed shown by an induction of E-cadherin protein expression and this was also seen during dual inhibition (FIG. 6B). Dual inhibition of HDAC6 and DHPS also showed a drastic nuclear accumulation of eIF5A in which TGFβ was not able to rescue shown by immunofluorescence (FIG. 6D).

To target this pathway, TubA was used to inhibit HDAC6 activity or Sirreal2 to inhibit SIRTUIN2 activity, as well as GC7 to inhibit DHPS. There are two alternative drugs available to target HDAC6, Tubacin and Ricolinostat—which is currently in clinical trials. An unexpected finding showed that dual inhibition further blocked TGFβ-induced EMT creating a method to block breast cancer progression and increase patient survival.

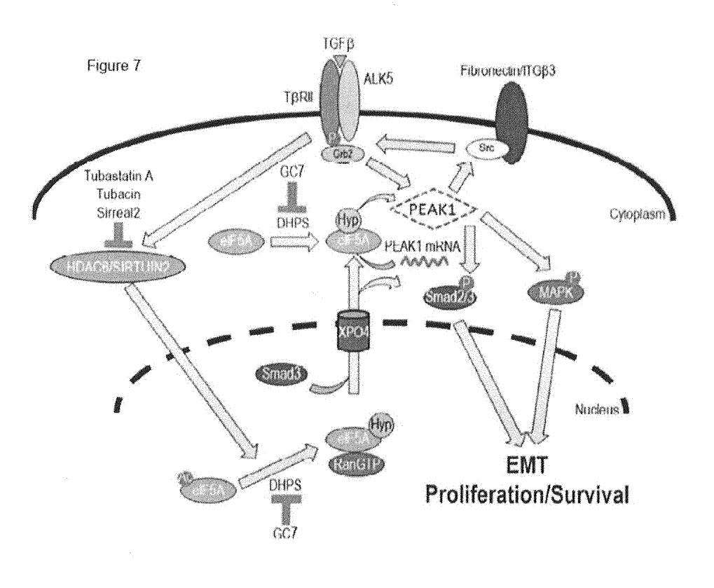

In sum, the inventors formulated a pathway in which TGFβ induces activation of HDAC6 or SIRTUIN2 to migrate into the nucleus to perform deacetylation of proteins such as eIF5A. Once eIF5A is deacetylated, DHPS and DOHH along with spermidine synthase activate eIF5A to form its hypusinated form. XPO4 recognizes its hypusine residue and shuttles eIF5A out into the cytoplasm for functioning. Many other proteins and mRNA are also shuttled out in this exportin molecule such as Smad3. PEAK1 mRNA can now be translated into protein where it can function in the tumor promoting role downstream of TGFβ. PEAK1 has been shown previously to activate Src in which Src phosphorylates TRII allowing for the recruitment of Grb2. Grb2 can then interact with PEAK1 to promote the activation of Smad2/3 and MAPK resulting in EMT (FIG. 7).

DHPS/SOX2/TP53 Axis is Associated with Diminished Patient Survival

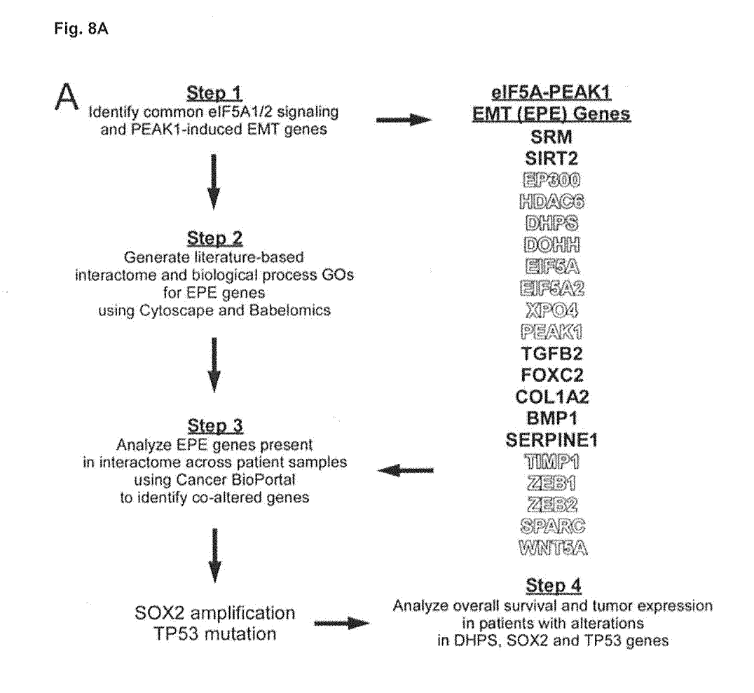

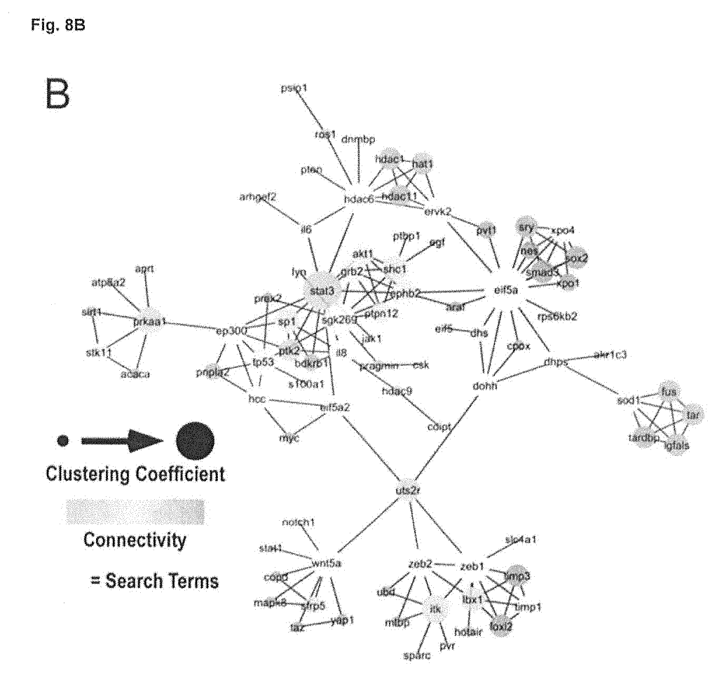

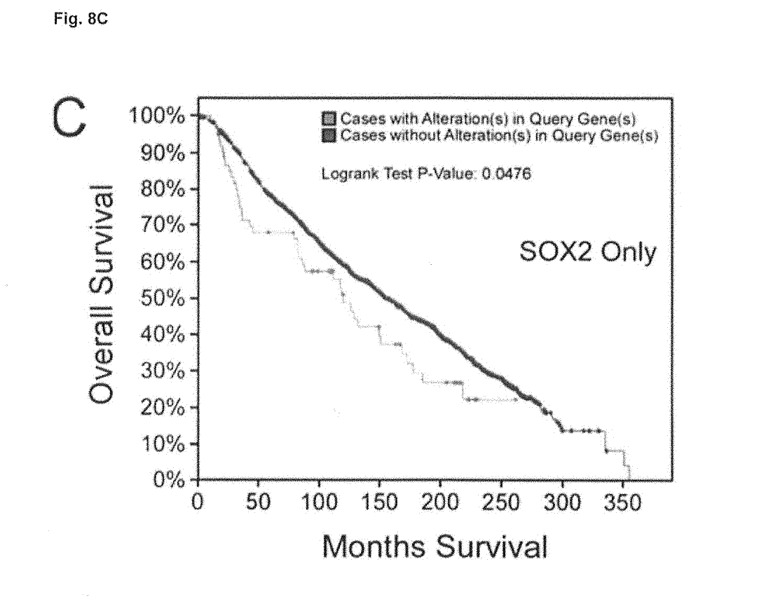

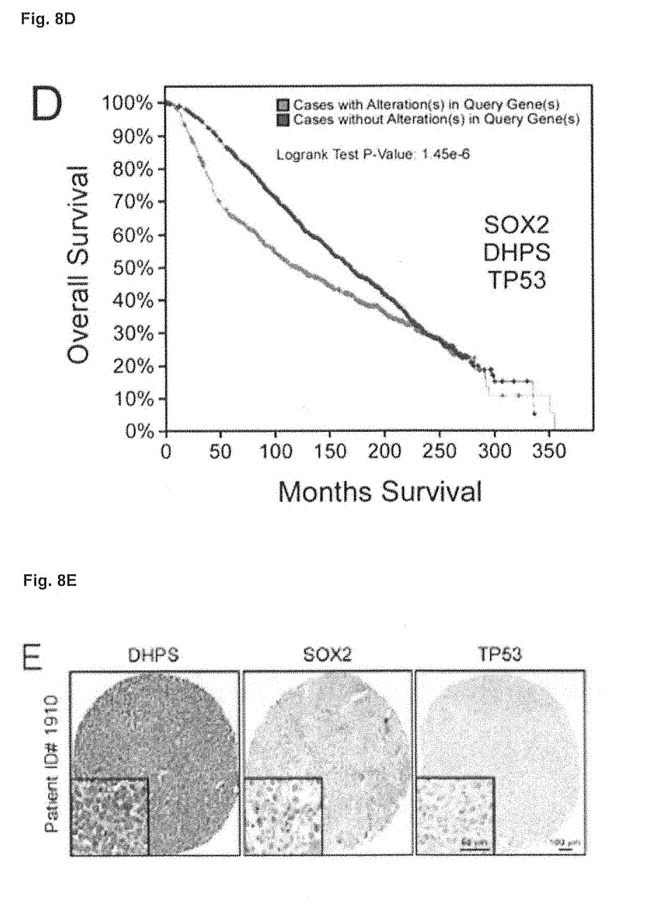

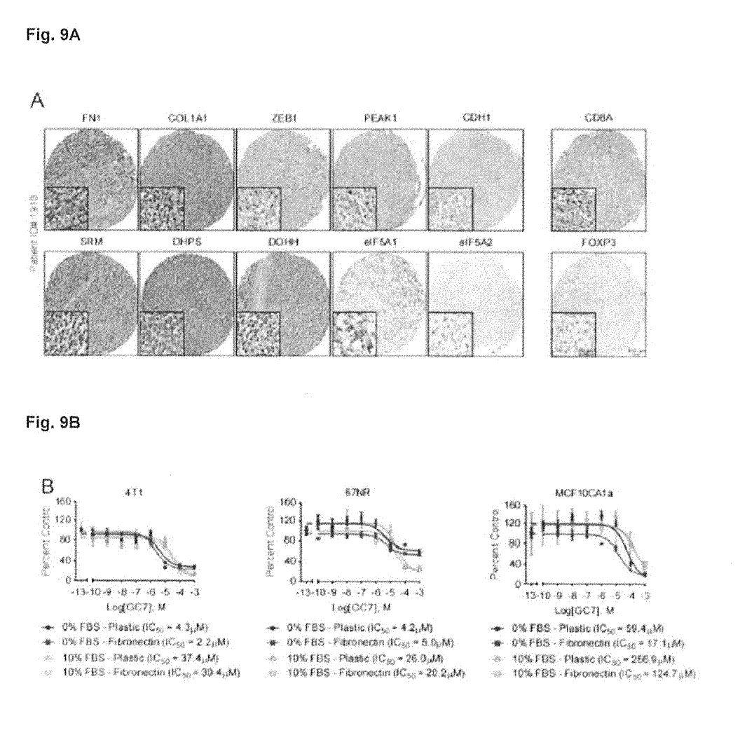

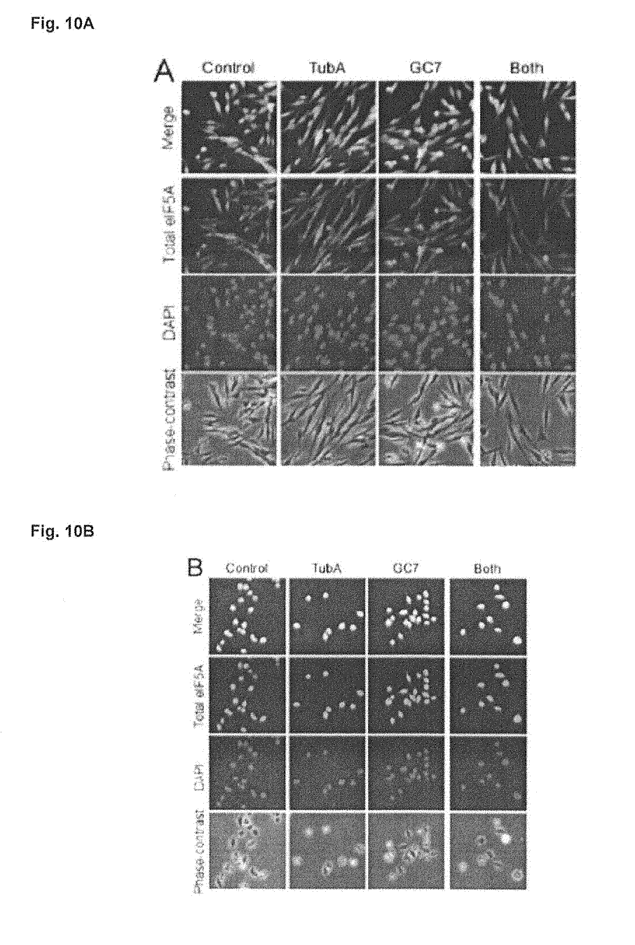

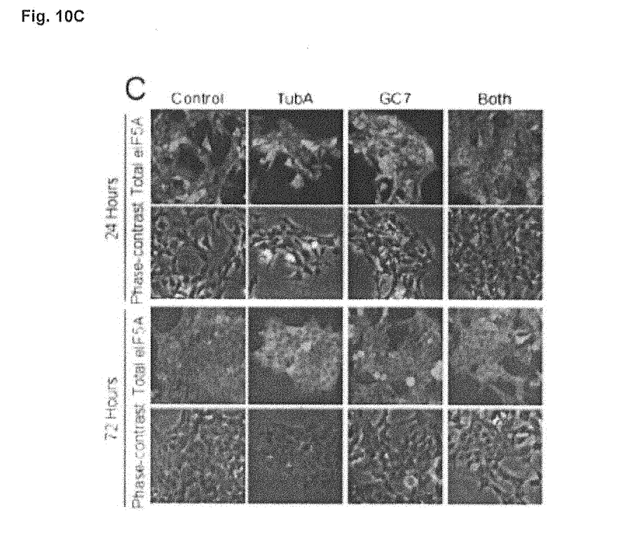

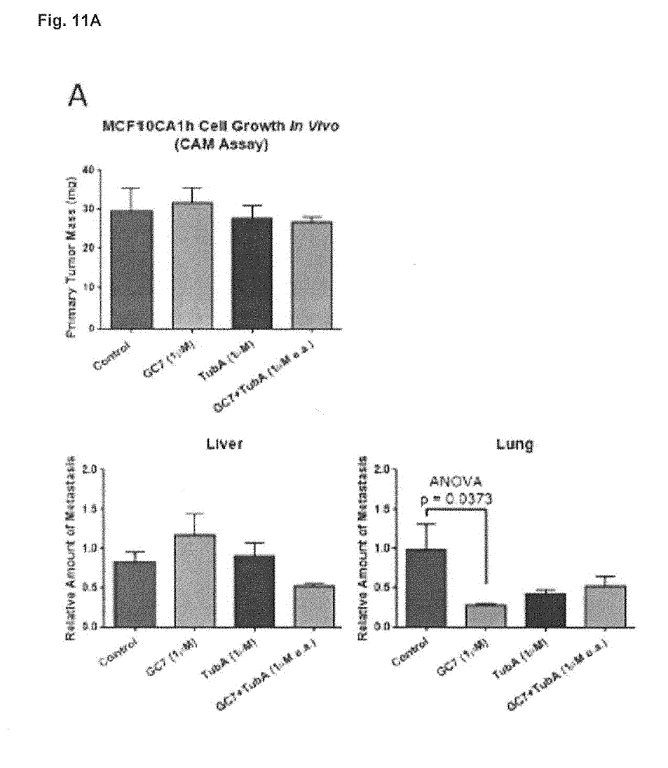

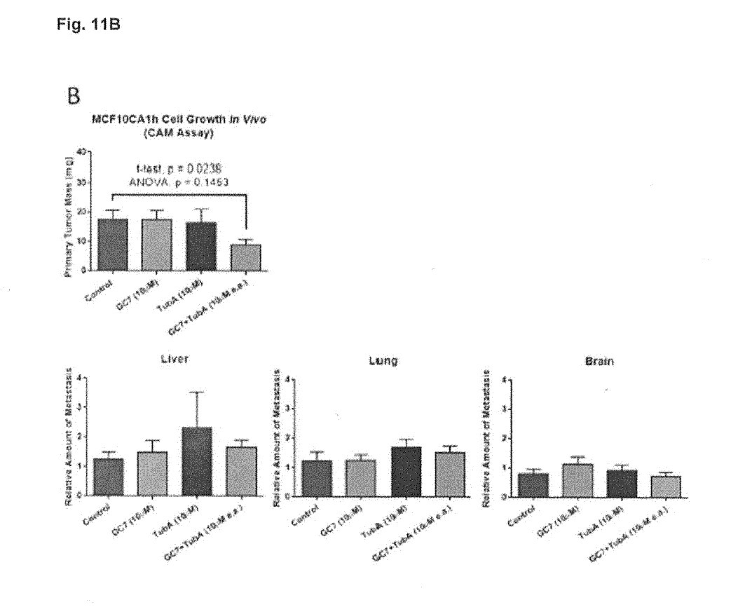

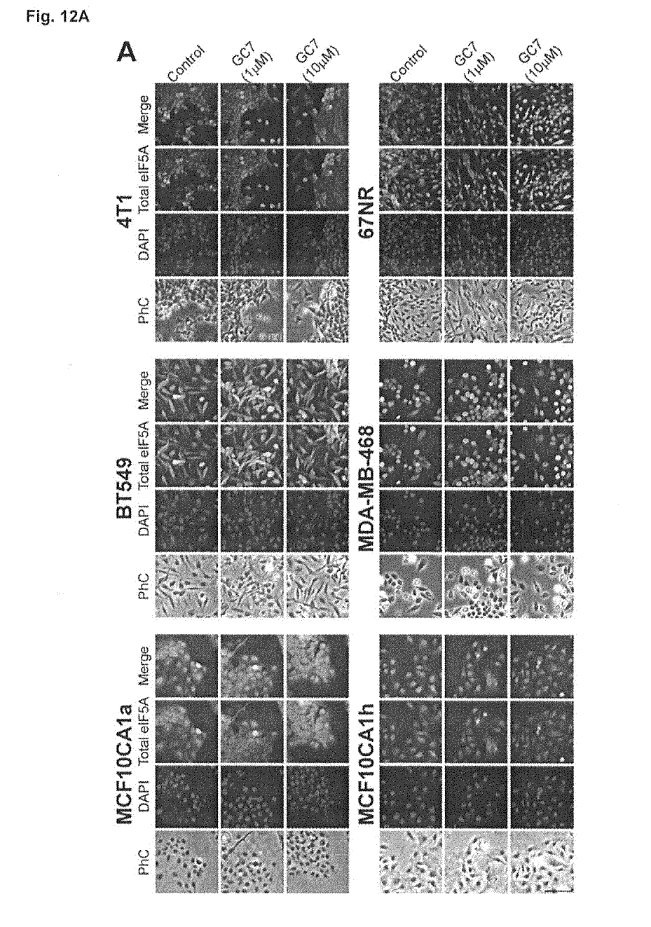

In order to assess whether there may be clinical significance to targeting the eIF5A hypusination-PEAK1 axis and related factors in breast cancer cells, we performed bioinformatics analyses to identify eIF5A and PEAK interactors and associated patient survival, as outlined in FIG. 8A. We first identified genes known to be involved in eIF5A function, as well as EMT genes whose expression has been shown to be affected by PEAK1 expression⁹. We used this list of genes to construct an interactome using Cytoscape (FIG. 8B). We also examined genes within the interactome for associated effects on patient survival using the METABRIC data on the cancer BioPortal^{52,56,57}. These analyses identified genetic amplifications in the stemness marker SOX2 and genetic mutations in the tumor suppressor gene p53 (TP53) as correlated the strongest with DHPS expression. These genes also contributed to diminished patient survival, as observed by the greatly reduced p-value in patients exhibiting modulations in all three genes compared to DHPS only (FIG. 8C, D). Immunohistochemically stained patient tumor data retrieved from the Human Protein Atlas confirmed the presence of DHPS and SOX2 proteins, while TP53 was undetectable (FIG. 8E). Additional protein components of the eIF5A-PEAK1 axis were found to be expressed in the same patient.

Material and Methods

Cell Culture

MCF10A, MCF10AT1k, MCF10AC1h and MCF10CA1a cells were purchased from the Karmanos Cancer Center (made in the laboratory of Dr. Fred Miller). MDA-MB-231, MDA-MB-468, 4T1, 67NR and BT549 cells were obtained from the American Tissue Culture Collection (ATCC). MCF10A and MCF10AT1k cells were cultured in Dulbecco's Modified Eagle's/Ham's Nutrient Mixture F-12 (DMEF12) growth media, supplemented with 5% horse serum, 10 μg/mL insulin, 20 ng/mL EGF, 0.5 μg/mL hydrocortisone, 100 ng/mL cholera toxin, 1% penicillin/streptomycin and 0.1% gentamycin. MCF10CA1h and MCF10CA1a cells were cultured in DMEF12 growth media and supplemented with 5% horse serum, 1% penicillin/streptomycin and 0.1% gentamycin. MDA-MB-231 and MDA-MB-468 cells were cultured in Dulbecco's Modified Eagle's Medium (DMEM)/High glucose growth media supplemented with 10% FBS, 1% penicillin/streptomycin and 0.1% gentamycin. 4T1 and 67NR cells were cultured in Rosewell Park Memorial Institute (RPMI) growth media supplemented with 10% FBS, 1% penicillin/streptomycin and 0.1% gentamycin. BT549 cells were also cultured in RPMI growth media supplemented with 10% FBS, 1% penicillin/streptomycin, 0.1% gentamycin and 0.8 μg/mL insulin. All cells were cultured in T25 flasks or 10 cm tissue culture dishes and grown at 37° C. with 5% CO₂.

Quantitative Polymerase Chain Reaction

Cell lines were plated in a 6 well plate at 1e5 cells/mL and left to attach overnight. Cells were then treated if necessary then trypsinized using 0.25% trypsin to collect whole cell pellets. RNA was collected using Thermo Scientific GeneJET RNA Purification Kit from all cell lines, reverse transcribed to cDNA using Thermo Scientific cDNA Synthesis Kit. Diluted cDNA at 22.5 ng/μL was used to perform qPCR. HPRT1 and POLAR2A were used as house-keeping genes. ABI 7300 system was used and set to the following three stages: stage 1 at 50° C. for 2 minutes for one replication. Stage 2 at 95° C. for 10 minutes for one replication and stage 3 at 95° C. for 15 seconds and 62° C. for 1 minute repeated for 40 replications. Cycle threshold (Ct) values were obtained once software was completed and used to determine relative mRNA expression of indicated gene targets used. All genes of interest were set relative to house-keeping genes.

Western Blot

Cell lines were plated in a 6 well plate at 1e5 cells/mL and left to attach overnight. Cells were then treated if necessary then lysed using Radioimmunoprecipitation assay (RIPA) buffer containing phosphatase and protease inhibitors and left to rotate at 4° C. for at least 3 hours. Lysates were then cleared by centrifugation at 12,000 RPM for 10 minutes at 4° C. Protein concentrations were determined using Bradford Assay. 4-12% Bis-Tris agarose gels were used to run protein lysates at 20 μg of protein that were stained with NuPage LDS Sample Buffer. Gels were then transferred onto nitrocellulose membranes and exposed to primary antibody solutions at the following concentrations for each indicated antibody. PEAK1 (Millipore 1:400), E-cadherin (Cell Signaling 1:1,000), Hypusine (Thermo Scientific 1:1,000), eIF5A (Thermo Scientific 1:1,000) and β-actin (Pro-Sci 1:1,000). Secondary antibodies were used at a 1:5,000-1:10,000 dilution. Band intensity graphs were quantified using Fiji software after image thresholding.

Cell Proliferation Assay

The CellTiter 96® AQueous One Solution (Promega) was used to perform cell proliferation experiments. Cells were plated at 5e3 cells/mL at 200 μL/well in a 96-well plate and allowed to attach overnight. Cells were then treated with indicated drugs the next day at various concentrations. After 72 hours of drug treatments, 40 μL of AQueous One solution was added to each well. Absorbance readings were measured at 490 nm wavelength at 1.5, 2 and 3 hours (absorbance is directly proportional to the number of living cells).

Immunofluorescence

Cells were plated at 1e4 cells/mL at 4 mL in a 6-well plate containing a sterile glass coverslip and allowed to attach overnight. Cells were then treated with appropriate inhibitors at indicated concentrations and time-points. Cells were first fixed using 4% paraformaldehyde for 20 minutes. Cells were then subjected to Triton-X to permeabilize the cells before treatment with specific antibodies in 2% BSA in PBS. After primary antibody incubation for 90 minutes, cells were washed with PBS three times before incubating with secondary antibodies in 2% BSA in PBS for 1 hour. Cells were again washed with PBS three times before set overnight staining with DAPI. Cells were then imaged using a Leica DMI6000 inverted microscope at 100× magnification.

Phase Contrast Micrographs

Images were taken of cells plated on a 6 well plate with appropriate conditions or treatments for experiment using widefield-brightfield phase contrast microscopy on a Leica DMI6000 B inverted microscope at 20× magnification.

TGFB Treatments

Cells were plated at 1e4 cells/mL or 1e5 cells/mL in a 6 well plate and left overnight to attach. The cells were then treated with 0.1% BSA or TGFβ at 2.5 ng/mL for times indicated in figures before collecting lysates or phase-contrast images.

Drug Treatments

Ciclopirox Olomine (CPX) was obtained by Santa Cruz Biotechnology at 10 mM stock concentration. This drug was diluted using Dimethyl Sulfoxide (DMSO). All controls used for CPX were DMSO. N1-Guanyl-1,7-diaminoheptane (GC7) was obtained by Biosearch Technologies at 100 mg stock concentration. This drug was diluted using sterile water. All controls used for GC7 were sterile water. Tubastatin A Hydrochloride (TubA) was obtained by Santa Cruz Biotechnology at 10 mM stock concentration. This drug was diluted using DMSO. All control used for TubA were DMSO.

Drug Treatments

The IHC human tissue samples shown during the current study are available in the Human Protein Atlas (http://www.proteinatlas.org) under patient #1910.

In sum, since eukaryotic initiation factor five A (eIF5A), a unique translation factor that is activated by post-translational hypusination, is required for PEAK1 expression, the inventors hypothesized that TGFβ may directly regulate eIF5A activity as a novel means of promoting EMT, and that targeting this pathway may help in the treatment of metastatic progression. In this regard, the inventors provide evidence of an active eIF5A-EMT program in undifferentiated breast cancer tissue. Notably, blockade of eIF5A hypusination (via deoxyhypusine synthase, DHPS, inhibition) reduces PEAK1 translation, cell viability and TGFβ-induced EMT in breast cancer cells. Conversely, the inventors demonstrate that TGFβ induces post-translational hypusination of eIF5A in metastatic breast cancer cells. TGFβ is known to activate histone deacetylase six (HDAC6) and HDAC6 was independently reported to promote eIF5A deacetylation and nuclear export, supporting its translation activity. When delivered in combination, HDAC6 and DHPS inhibitors synergize to sequester eIF5A to the nucleus, suppress eIF5A-dependent translation and potently kill metastatic breast cancer cells. To identify additional pathways downstream of eIF5A during EMT, the inventors generated a Cytoscape interactome using eIF5A signaling and PEAK1-induced EMT genes as search terms. Genes from the parent list in the resulting interactome were analyzed in two breast cancer studies available on the Cancer Bio Portal. Interestingly, SOX2, PIK3CA and EIF4A2 were the genes that exhibited copy number amplifications among patients harboring alterations in our search list genes, while SOX2 was the only candidate that significantly and independently associated with decreased patient survival (p=0.0476). Taken together, these results establish a signaling pathway by which TGFβ stimulates HDAC6 and DHPS function to activate cytoplasmic eIF5A and promote EMT and survival in breast cancer cells.

Targeting DHPS to Abrogate TGFB-Induced Metastasis in Breast Cancer

Progression of solid tumors to a metastatic stage accounts for over 90% of cancer mortality. Thus, it is critical to identify therapeutic strategies that target both primary and metastatic tumors. Epithelial-mesenchymal transition (EMT) negatively correlates with therapy response, contributing to intratumoral heterogeneity and systemic dissemination in breast cancer. We previously reported that pseudopodium-enriched atypical kinase one (PEAK1) promotes breast cancer cell EMT and metastasis by potentiating fibronectin-transforming growth factor beta (TGFβ) signaling cross-talk. Since eukaryotic initiation factor five A (eIF5A), a unique translation factor that is activated by deoxyhypusine synthase (DHPS)-dependent post-translational hypusination, is required for PEAK1 expression, we hypothesized that TGFβ may directly regulate eIF5A activity to promote EMT, and that targeted inhibition of this pathway may provide a novel means to inhibit or reverse metastatic progression. In this regard, we provide evidence of an active eIF5A-EMT program in undifferentiated breast cancer tissue. Notably, blockade of DHPS activity and eIF5A hypusination reduces PEAK1 translation, cell viability and TGFβ-induced EMT in vitro and metastasis in vivo. Conversely, we demonstrate that TGFβ induces post-translational hypusination of eIF5A in metastatic breast cancer cells. TGFβ is known to activate histone deacetylase six (HDAC6) and HDAC6 was independently reported to promote eIF5A deacetylation and nuclear export to support its translation functions. When delivered in combination, HDAC6 and DHPS inhibitors synergize to sequester eIF5A to the nucleus, suppress eIF5A-dependent translation and potently kill metastatic breast cancer cells. To identify candidate pathways downstream of the eIF5A/PEAK1 axis during EMT, we generated a Cytoscape interactome using eIF5A signaling and PEAK1-induced EMT genes as search terms. All interactome component genes were then analyzed across two breast cancer patient studies available on the Cancer BioPortal. Interestingly, SOX2, PIK3CA and EIF4A2 were the interactome nodes that exhibited copy number amplifications among patients harboring genomic alterations in the initial interactome search genes, and SOX2 amplification significantly and independently associated with decreased patient survival (p=0.0476). Taken together, our results establish a novel node/axis by which TGFβ signaling stimulates HDAC6 and/or DHPS function to activate cytoplasmic eIF5A and promote EMT and survival of breast cancer cells within the metastatic niche—identifying new targeted therapy strategies that may improve cancer patient survival.

Thus, specific embodiments, methods of compositions and methods for the treatment and prevention of cancer have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

All references cited herein, including those below and including but not limited to all patents, patent applications, and non-patent literature referenced below or in other portions of the specification, are hereby incorporated by reference herein in their entirety.
1. Breastcancer.org
2. Wahba H A and El-Hadaad H A. “Current Approaches in Treatment of Triple-Negative Breast Cancer.” Cancer Biol Med 2015; 12(2):106-16.
3. Siegel et al. “Cancer Statistics, 2016”. CA Cancer J Clin 2016; 66(1):7-30.
4. Wang and Kelber et al. “Pseudopodium-enriched Atypical Kinase 1 Regulated the Cytoskeleton and Cancer Progression.” PNAS, 2010; 107(30):13556.
5. Wang et al. “Methods for Pseudopodia Purification and Proteomic Analysis”. Sci STKE 2007; (400):p14.
6. Kelber J A and Klemke R L. “PEAK1, a Novel Kinase Target in the Fight Against Cancer”. Oncotarget 2010; 1(3):219-223.
7. Zody et al. “DNA Sequence of Human Chromosome 17 and Analysis of Rearrangement in the Human Lineage”. Nature 2006; 440(7087):1045-9.
8. Kelber et al. “KRas Induces a Src/PEAK1/ErbB2 Kinase Amplification Loop that Drives Metastatic Growth and Therapy Resistance in Pancreatic Cancer.” Cancer Research. 2012; 72(10):2554-64.
9. Croucher et al. “Involvement of Lyn and the Atypical Kinase Sgk269/PEAK1 in a Basal Breast Cancer Signaling Pathway”. Cancer Research 2013; 73(6):1969-80.
10. Agajanian et al. “PEAK1 Acts as a Molecular Switch to Regulate Context-Dependent TGFβ Responses in Breast Cancer.” PLoS One. 2015; 10(8):e0135748.
11. Agajanian et al. “Identification of a PEAK1/ZEB1 Signaling Axis during TGFβ/Fibronectin-Induced EMT in Breast Cancer.” BBRC. 2015; 465(3):606-12.
12. Zheng et al. “Temporal Regulation of EGF Signaling Networks by the Scaffold Protein Shc1”. Nature 2013; 499(7457):166-71.
13. Fujimura et al. “A Hypusine-eIF5A-PEAK1 Switch Regulates the Pathogenesis of Pancreatic Cancer.” Cancer Research. 2014; 74(22):6671-81.
14. Runa F, Adamian Y and Kelber J A. “Ascending the PEAK1 Toward Targeting TGFβ During Cancer Progression: Recent Advances and Future Perspectives”. CCM 2016; 3:e1162.
15. Massague J. “TGFbeta in Cancer”. Cell 2008; 134(2):215-30.
16. Galliher-Beckley A J and Schieman W P. “Grb2 Binding to Tyr284 in TbetaR-II is Essential for Mammary Tumor Growth and Metastasis Stimulated by TGF-beta”. Carcinogenesis 2008; 29(2):244-51.
17. Galliher-Beckley A J and Schieman W P. “Src Phosphorylates Tyr284 in TbetaR-II and Regulates TGF-beta Stimulation of p38 MAPK During Breast Cancer Cell Proliferation and Invasion”. Cancer Res. 2007; 67(8):3752-8.
18. Uhlen M et al. “Towards a Knowledge-based Human Protein Atlas”. Nat Biotechnol 2010; 28(12):1248-50.
19. Shah A A et al. “Identification of a Selective SIRT2 Inhibitor and its Anti-Breast Cancer Activity” Biol Pharm Bull 2016; 39(10):1739-1742.
20. Gu S et al. “Loss of Alpha-Tubulin Acetylation is Associated with TGFβ-Induced Epithelial-Mesenchymal Transition” JBC 2016; 291(10):5396-405.
21. Ishfaq M et al. “The Role of Acetylation in the Subcellular Localization of an Oncogenic Isoform of Translation Factor eIF5A” Biosci Biotechnol Biochem 2012; 76(11):2165-7.
22. Aksu M et al. “Structure of the Exportin XPO4 in Complex with RanGTP and the Hypusine-Containing Translation Factor eIF5A” Nat Commun 2016; 16(7):11952.
23. Kurisaki A et al. “The Mechanism of Nuclear Export of Smad3 Involves Exportin 4 and Ran” Mol Cell Biol 2006; 26(4):1318-32.
24. Liposwky G et al. “Exportin 4: A Mediator of a Novel Nuclear Export Pathway in Higher Eukaryotes” EMBO J 2000; 19(16):4362-71.
25. Schuller A P et al. “eIF5A Functions Globally in Translation Elongation and Termination” Mol Cell 2017; 2765(17)30170-3.
26. Mandal A et al. “Global Quantitative Proteomics Reveal Up-Regulation of Endoplasmic Reticulum Stress Response Proteins Upon Depletion of eIF5A in HeLa Cells” Sci Rep 2016; 16(6):25795.
27. Sato M et al. “Differential Proteome Analysis Identifies TGFβ-Related Pro-Metastatic Proteins in a 4T1 Murine Breast Cancer Model” PLos One 2015; 10(5):e0126483.
28. Imam S et al. “Eukaryotic Translation Initiation Factor 5A Inhibition Alters Physiopathology and Immune Responses in a ‘Humanized’ Transgenic Mouse Model of Type 1 Diabetes” Am J Physiol Endocrinol Metab 2014; 306(7):E791-E798.
29. Jenkins Z A et al. “Human eIF5A2 on Chromosome 3q25-q27 is a Phylogenetically Conserved Vertebrate Variant of Eukaryotic Translation Initiation Factor 5A with Tissue-Specific Expression” Genomics 2001; 71:101-109.
30. Cracchiolo B M et al. “Eukaryotic Initiation Factor 5A-1 (eIF5A-1) as a Diagnostic Marker for Aberrant Proliferation in Intraepithelial Neoplasia of the Vulva” Gynecologic Oncology 2004; 92(1):217-222.
31. Taylor C A et al. “Eukaryotic Translation Initiation Factor 5A Induces Apoptosis in Colon Cancer Cells and Associates with the Nucleus in Response to Tumour Necrosis Factor Alpha Signaling” Experimental Cell Research 2007; 313(3):437-449.
32. Hu X, Huang W and Fan M. “Emerging Therapies for Breast Cancer” Journal of Hematology & Oncology 2017; 10:98.
33. Loi S et al. “Prognostic and Predictive Value of Tumor-Infiltrating Lymphocytes in a Phase III Randomized Adjuvant Breast Cancer Trial in Node-Positive Breast Cancer Comparing the Addition of Docetaxel to Doxorubicin with Doxorubicin-Based Chemotherapy: BIG 02-98” J Clin Oncol 2013; 31(7):860-7.
34. Mahmoud S M et al. “Tumor-Infiltrating CD8+ Lymphocytes Predict Clinical Outcome in Breast Cancer” J Clin Oncol 2011; 29:1949-1955.
35. Mittendorf E A et al. “PD-L1 Expression in Triple Negative Breast Cancer” Cancer Immunol Res 2014; 2(4):361-70.
36. Muenst S et al. “Expression of Programmed Death Ligand 1 (PD-L1) is Associated with Poor Prognosis in Human Breast Cancer” Breast Cancer Res Treat. 2014; 146(1):15-24.
37. Colvin S C et al. “Deoxyhypusine Synthase Promotes Differentiation and Proliferation of T Helper Type 1 (Th1) Cells in Autoimmune Diabetes” Journal of Biological Chemistry. 2013; 288:36226-36235.
38. Kalluri R and Weinberg R A. “The Basics of Epithelial-Mesenchymal Transition” Journal of Clinical Investigations 2009; 119(6):1420-1428.
39. Thiery J P. “Epithelial-Mesenchymal Transition in Tumour Progression” Nat Rev Cancer 2002; 2(6):442-54.
40. Thiery J P and Huang R. “Linking Epithelial-Mesenchymal Transition to the Well-Known Polarity Protein Par6” 2005; 8(4):456-8.

### ADDITIONAL RELATED REFERENCES

41. Nakanishi S, Cleveland J L. Targeting the polyamine-hypusine circuit for the prevention and treatment of cancer. Amino Acids 2016; 48: 2353-2362.
42. Zuk D, Jacobson A, Lamond A, Solsbacher J, Kutay U, Görlich D et al. A single amino acid substitution in yeast eIF-5A results in mRNA stabilization. EMBO J 1998; 17: 2914-2925.
43. Lee S B, Park J H, Kaevel J, Sramkova M, Weigert R, Park M H. The effect of hypusine modification on the intracellular localization of eIF5A. Biochem Biophys ResCommun 2009; 383: 497-502.
44. Zhang J, Li X, Liu X, Tian F, Zeng W, Xi X et al. EIF5A1 promotes epithelial ovarian cancer proliferation and progression. Biomed Pharmacother 2018; 100: 168-175.
45. Chen C, Zhang B, Wu S, Song Y, Li J, Wu S et al. Knockdown of EIF5A2 inhibits the malignant potential of non-small cell lung cancer cells. Oncol Lett 2018; 15: 4541-4549.
46. He L-R, Zhao H-Y, Li B-K, Liu Y-H, Liu M-Z, Guan X-Y et al. Overexpression of eIF5A-2 is an adverse prognostic marker of survival in stage I non-small cell lung cancer patients. Int J cancer 2011; 129: 143-50.
47. Tunca B, Tezcan G, Cecener G, Egeli U, Zorluoglu A, Yilmazlar T et al. Overexpression of CK20, MAP3K8 and EIF5A correlates with poor prognosis in early-onset colorectal cancer patients. J Cancer Res Clin Oncol 2013; 139: 691-702.
48. Guan X-Y, Fung J M-W, Ma N-F, Lau S-H, Tai L-S, Xie D et al. Oncogenic role of eIF-5A2 in the development of ovarian cancer. Cancer Res 2004; 64: 4197-200.
49. Tang D-J, Dong S-S, Ma N-F, Xie D, Chen L, Fu L et al. Overexpression of eukaryotic initiation factor 5A2 enhances cell motility and promotes tumor metastasis in epatocellular carcinoma. Hepatology 2010; 51: 1255-1263.
50. Liu Y, Du F, Chen W, Yao M, Lv K, Fu P. EIF5A2 is a novel chemoresistance gene in breast cancer. Breast Cancer 2015; 22: 602-607.
51. David C J, Massagué J. Contextual determinants of TGFβ action indevelopment, immunity and cancer. Nat Rev Mol Cell Biol 2018; 19: 419-435.
52. Pereira B, Chin S-F, Rueda O M, Vollan H-K M, Provenzano E, Bardwell H A et al. The somatic mutation profiles of 2,433 breast cancers refines their genomic and transcriptomic landscapes. Nat Commun 2016; 7: 11479.
53. Uhlen M, Zhang C, Lee S, Sjöstedt E, Fagerberg L, Bidkhori G et al. A pathology atlas of the human cancer transcriptome. Science 2017; 357: eaan2507.
54. Uhlen M, Fagerberg L, Hallstrom B M, Lindskog C, Oksvold P, Mardinoglu A et al. Tissue-based map of the human proteome. Science (80-) 2015; 347: 1260419-1260419.
55. Shannon P, Markiel A, Ozier O, Baliga N S, Wang J T, Ramage D et al. Cytoscape: a software environment for integrated models of biomolecular interaction networks. Genome Res 2003; 13: 2498-504.
56. Cerami E, Gao J, Dogrusoz U, Gross B E, Sumer S O, Aksoy B A et al. The eBio cancer genomics portal: an open platform for exploring multidimensional cancer genomics data. Cancer Discov 2012; 2: 401-4.
57. Gao J, Aksoy B A, Dogrusoz U, Dresdner G, Gross B, Sumer S O et al. Integrative analysis of complex cancer genomics and clinical profiles using the cBioPortal. Sci Signal 2013; 6: p11.

What is claimed is:

1. A method of treating a subject with cancer comprising administering to a subject in need thereof an effective amount of a pharmaceutical composition comprising at least one inhibitor of histone deacetylase six (HDAC6) and an inhibitor of deoxyhypusine synthase (DHPS); at least one inhibitor of histone deacetylase six (HDAC6) or an inhibitor of deoxyhypusine synthase (DHPS), or a combination thereof.

2. The method of claim 1, wherein the cancer is breast cancer or pancreatic cancer.

3. The method of claim 1, wherein the at least one inhibitor of HDAC6 comprises: CAY10603, Tubacin, Ricolinostat (ACY-1215), Nexturastat A, Tubastatin A HCl, Tubastatin A, HPOB, CUDC-101, PCI-24781 (Abexinostat), CUDC-907, Resminostat, Quisinostat (JNJ-26481585), Pracinostat (SB939), Droxinostat, PCI-34051, or a combination thereof.

4. The method of claim 2, wherein the at least one inhibitor of DHPS comprises:

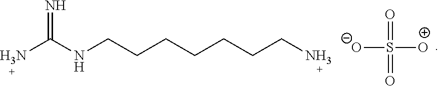 [SMILES: N=C([NH3+])NCCCCCCC[NH3+] ; O=S(=O)([O-])[OH2+]]

5. The method of claim 2, wherein the at least one inhibitor of HDAC6 comprises: CAY10603, Tubacin, Ricolinostat (ACY-1215), Nexturastat A, Tubastatin A HCl, Tubastatin A, HPOB, CUDC-101, PCI-24781 (Abexinostat), CUDC-907, Resminostat, Quisinostat (JNJ-26481585), Pracinostat (SB939), Droxinostat, PCI-34051, or a combination thereof, and wherein the at least one inhibitor of DHPS comprises:

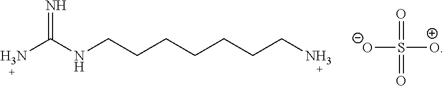

6. The method of claim 5, wherein the inhibitor of HDAC6 is Tubastatin A, and wherein the inhibitor of DHPS is GC7 (CAS 150333-69-0).

7. A pharmaceutical composition for the treatment of cancer comprising at least one inhibitor of histone deacetylase six (HDAC6) and an inhibitor of deoxyhypusine synthase (DHPS); at least one inhibitor of histone deacetylase six (HDAC6) or an inhibitor of deoxyhypusine synthase (DHPS), or a combination thereof.

8. The pharmaceutical composition of claim 7, wherein the cancer is breast cancer or pancreatic cancer.

9. The pharmaceutical composition of claim 7, wherein the at least one inhibitor of HDAC6 comprises: CAY10603, Tubacin, Ricolinostat (ACY-1215), Nexturastat A, Tubastatin A HCl, Tubastatin A, HPOB, CUDC-101, PCI-24781 (Abexinostat), CUDC-907, Resminostat, Quisinostat (JNJ-26481585), Pracinostat (SB939), Droxinostat, PCI-34051, or a combination thereof, and wherein the inhibitor of DHPS is:

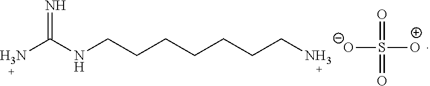 [SMILES: N=C([NH3+])NCCCCCCC[NH3+] ; O=S(=O)([O-])[OH2+]]

10. The pharmaceutical composition of claim 9, wherein the at least one inhibitor of HDAC6 is Tubastatin A, and wherein the inhibitor of DHPS is GC7 (CAS 150333-69-0).

11. The pharmaceutical composition of claim 10, further comprising at least one excipient.

12. A method of treating a subject with cancer comprising administering to a subject in need thereof an effective amount of a pharmaceutical composition comprising at least one inhibitor of sirtuin and an inhibitor of deoxyhypusine synthase (DHPS); at least one inhibitor of sirtuin or an inhibitor of deoxyhypusine synthase (DHPS), or a combination thereof.

13. The method of claim 12, wherein the cancer is breast cancer or pancreatic cancer.

14. The method of claim 12, wherein sirtuin is sirtuin-2.

15. A method of treating a subject with cancer comprising administering to a subject in need thereof an effective amount of a pharmaceutical composition comprising at least one inhibitor of sirtuin and at least one inhibitor of histone deacetylase six (HDAC6); at least one inhibitor of sirtuin or at least one inhibitor of histone deacetylase six (HDAC6), or a combination thereof.

16. The method of claim 15, wherein the cancer is breast cancer or pancreatic cancer.

17. The method of claim 15, wherein sirtuin is sirtuin-2.

18. A method of treating a subject with cancer comprising administering to a subject in need thereof an effective amount of a pharmaceutical composition comprising at least one inhibitor of sirtuin, an inhibitor of deoxyhypusine synthase (DHPS), and at least one inhibitor of histone deacetylase six (HDAC6); at least one inhibitor of sirtuin, an inhibitor of deoxyhypusine synthase (DHPS), or at least one inhibitor of histone deacetylase six (HDAC6), or a combination thereof.

19. The method of claim 18, wherein the cancer is breast cancer or pancreatic cancer.

20. The method of claim 18, wherein sirtuin is sirtuin-2.
